(19) United States
(12) Patent Application Publication
(10) Pub. No.: US 2014/0158604 A1
(43) Pub. Date: Jun. 12, 2014

(54) Platelet Storage Container

(71) Applicant: Jacques Chammas, Walpole, MA (US)

(72) Inventors: Marie Joelle Chammas, Walpole, MA (US); Jacques Chammas, Walpole, MA (US)

(73) Assignee: Jacques Chammas, Walpole, MA (US)

(21) Appl. No.: 14/105,045

(22) Filed: Dec. 12, 2013

Related U.S. Application Data
(63) Continuation-in-part of application No. 13/711,613, filed on Dec. 12, 2012.

(51) Int. Cl. A61M 1/36; A61M 1/16

(52) U.S. Cl. CPC A61M 1/3679; A61M 1/1621
USPC 210/256

(57) ABSTRACT

Compositions, methods, devices and media are provided for platelet storage container that prevents bacterial growth in the stored platelets. The invention relates to blood bank needs in safely storing platelets collected from donors for more than 5 days at room temperature. The container is built with natural adsorbent media that have the characteristics in capturing and killing bacteria and viruses. The stored platelets are isolated from the natural adsorbent material during storage to preserve their medical quality and safety.

### FIELD OF THE INVENTION

This invention relates to an improved RBC, leukocyte, or platelet storage method and composition. More particularly, this invention aims to minimize pathogen contamination of the stored platelet concentrates and of packed platelets suitable for transfusion. This invention also presents a new approach that neutralizes the effect of the citric anticoagulant in the re-infused blood products.

### BACKGROUND OF THE INVENTION

Platelets are small cellular (2 μm to 4 μm) megakaryocytes components of the blood that provide primary hemostasis function that leads to the stop of bleeding. Platelets transfusion is an established medical therapy used to help patient's recovery post major surgeries or post chemotherapy treatments. Platelets are either derived from collected units of human whole blood using special hematology techniques or are directly collected from a healthy human donor using special apheresis systems.

In routine blood banking practice, human platelet concentrates (PC) are prepared by drawing a unit of blood (about 450 ml) into a plastic bag containing an anticoagulant and then centrifuging the blood into three fractions: red cells, plasma, and platelets. The separated platelet fraction is then suspended in approximately 50 ml of plasma. This platelet-containing product is then stored until needed for transfusion into a patient.

After platelets collection they are stored for up to 5 days to be infused in patients. Platelets are stored in bags made of plastic film material with high O₂ and CO₂ permeability to control platelet metabolism and pH level. In order for the platelets to maintain their function for reinfusion, they are stored under constant agitation at 22° C. This storage temperature provides the environment for any viruses or bacteria that are introduced to the blood during the collection process to proliferate and subsequently may cause sepsis complications to the recipient.

Bacterial contamination of platelets represents the most frequent transfusion-associated infectious risk. All blood components are susceptible to bacterial contamination. Unlike the plasma and the red blood cells (RBC) that are stored at low temperature, the platelets are stored at room temperature to preserve its function. By storing platelets at temperature ranging between 20° C. and 24° C., bacteria proliferation becomes a realistic complication. Skin flora is the most common source of bacterial contamination during blood collection. The estimated bacterial load at collection is less than 0.1 CFU/ml. These small bacterial inoculums can proliferate within a short time period to serious and substantial levels in platelet concentrate that are stored at room temperature. Bacterial contamination of platelets can be found in about 1 in 1,000 to 1 in 3,000 units.

In addition to storage time, other storage conditions have been shown to affect platelet metabolism and function. Initial pH, storage temperature, total platelet count, plasma volume, agitation during storage, and hydrogen ion accumulation are some of the factors known to influence the storage of platelets.

A number of other interrelated variables can also affect platelet viability and function during storage, namely, the anticoagulant used for blood collection, the method used to prepare platelet concentrates and the composition, surface area, and thickness of the walls of the storage container.

One of the major problems in PC storage is regulation of pH. Virtually all units of PC show a decrease in pH from their initial value of approximately 7.0. This decrease is primarily due to the production of lactic acid by platelet glycolysis and to a lesser extent to accumulation of CO₂ from oxidative phosphorylation.

As pH level in the stored platelets bag falls from 6.8 to 6.0, the platelets progressively change shape from discs to spheres. In this pH range, the change of shape is reversible if the platelets are resuspended in plasma with physiologic pH. However, if the pH falls below 6.0, a further irreversible change occurs which renders the platelets nonviable after infusion in vivo. Oxygen supply to the platelets within the plastic bag is also intimately related to pH maintenance. If the supply is sufficient, glucose will be metabolized oxidatively resulting in CO₂ production, which diffuses out of the plastic walls of the PC container. If the supply of oxygen is insufficient, glucose will be metabolized anaerobically, resulting in the production of lactic acid, which must remain within the container and thus lowers the pH. The oxygen tension within the container is governed by several factors: the concentration of platelets which consume oxygen, the permeability of the plastic wall of the PC, the surface area of the container available for gas exchange, and the type of agitation utilized.

The present goal of platelet preservation is to prevent this change in pH and to minimize the pathogens growth in the storage media. There are many attempts in the prior arts to prevent the change in the pH and to eradicate all pathogens. All these methods were founded on chemical and/or radiation treatments of the blood products. Chemicals that proved to be effective in eradicating bacteria and viruses could pose serious side effects on blood cells such as red blood cells (RBC) and platelets.

The object of the current invention is to provide a passive method to prevent unwanted changes in pH and to minimize the bacteria and viruses growth in the blood product storage environment. Whereas the passive method in this invention is not dependent on mixing chemicals with blood products nor utilizes any type of radiation to treat blood products.

### DISCLOSURE OF THE INVENTION

The current invention aims to minimize bacterial contamination of the stored platelets. More specifically to minimize bacterial contamination inside the platelet bag during storage. Therefore, diminishing the risk of septicemia acquired in the course of platelets transfusion. In this invention, the platelet bag is designed to incorporate a sachet that contains adsorbent media. The adsorbent media has inherent tendency to capture bacteria and viruses from solutions. These captured bacteria and viruses are prevented from being mixed with the environment that contains plasma and platelets. The media that is contained inside the sachet is safe when it comes in contact with plasma but in some cases it might cause platelet adhesion or red blood cell Hemolysis when it comes in contact with blood cells. Therefore, it is important to keep the blood cells from contacting the adsorbent media.

For convenience all bacteria, micro-organisms, viruses, cytokines, endotoxins, and toxin will be referred to in this study as pathogens.

The sachet that contains the media is made of biocompatible membrane with porosity that prevents any blood cell from passing through while bacteria and viruses suspended in the plasma can pass freely through the membrane. Typically bacteria are 1 μm (micron) or less in size. Viruses are less than 1 μm in size. Platelets are (2 μm to 4 μm) in size and red blood cells (RBC) are 7 μm in size. Platelets usually have a disc shape that could transform to a spherical shape at cold temperatures. RBCs have a disc shape with 7 μm in diameter and 2 μm thick. White blood cells have a spherical shape with 10 μm in diameter.

Platelets with bacteria and viruses are suspended in the plasma inside the platelet bag. The sachet containing adsorbent media is located inside the platelet bag. Platelets cannot penetrate the sachet membrane and they cannot come in contact with the adsorbent media inside the sachet. Platelets are confined in the space inside the platelet bag but outside the sachet. Bacteria and viruses are floating in the plasma can easily penetrate the sachet membrane. Therefore, the bacteria, viruses, and plasma are located inside the whole space defined by the platelet bag including the sachet. As bacteria and viruses cross through the sachet membrane and come in contact with the adsorbent media inside, they stick to the media surface and become trapped. The majority of the bacteria and the viruses that flow inside the sachet adhere to the adsorbent surface and are prevented from becoming loose. Therefore, they are entrapped inside the sachet and do not intermix with the platelets that float in the plasma outside the sachet. In addition, cytokines, endotoxins, and other micro-organisms and toxins that are suspended in the plasma; are also trapped by the activated carbon inside the sachet. U.S. Pat. No. 6,852,224 by Jagtoyen et al. discloses a filter comprised of activated carbon fibers, wherein said filter has a Virus Removal Index (VRI) of at least about 99%, as measured in accordance with the test method described in the specification. U.S. Pat. No. 4,898,676 by Horowitz discloses a method of using grafted activated carbon to remove bacteria from contaminated water. U.S. Pat. No. 6,989,101 by Cumberland et al. discloses activated carbon media filter for the removal of micro-organism from a medium. There are many research papers and published literature and books supporting the concept of pathogen adsorption by activated carbon and by porous polymeric resins of Styrenic matrix.

When platelets are needed for re-infusion, they are pumped out of the bag with the plasma by gravity or by a pump such as peristaltic pump. The bacteria and viruses that crossed the sachet membrane and were absorbed by the media inside remain in the bag. Therefore, a lesser number of bacteria and viruses are mixed with the platelets when exiting the bag for re-infused. It is obvious that the more bacteria and viruses are absorbed by the media, the smaller their number is with the infused platelet product.

In most blood centers the bag containing the platelets is laid down on a shaker that is continuously agitated at a rate of 70 to 80 cycles per minute. The oscillation movement of the shaker flushes the plasma back and forth inside the bag. This flushing movement generates plasma current that flows in and out the sachet through the porous membrane. Bacteria, micro-organisms, viruses, cytokines, endotoxins, lipids, proteins, and other electrolytes flow through the porous membrane with the plasma, while the platelets and other cells are kept out of the sachet.

In a preferred embodiment the media contained in the sachet is made of activated carbon cloth having large surface area (700-2000 m²/g) being predominately microporous, that are commercially available. Bundles of very fine activated carbon fibers are used to weave or knit carbon cloth. The cloth can also be impregnated with chemical treatments to make more sensitive to adsorption of particular molecules. Electrostatic forces could be developed within the cloth to enhance its adsorbing efficiency. The cloth can be woven or knitted with different weights and thickness using fibers, nano-fibers, or nano tubes carbon. The cloth can be made out of felt with different weights and thickness. The media could also be made of particulate (beads, graduals) activated carbons sold by Calgon Carbon Corp. or Norit Americas Inc. (Marshall, Tex., USA). The media could also be made of activated carbon fibers and microfibers sold by Kureha (Tokyo, Japan), nano-fibers, or nano-tubes.

Activated carbon (AC) is adsorbent that is manufactured from a carbon based material. Some of the common carbonaceous substances used as raw materials to make activated carbon are coal lignite, sub-bituminous, and bituminous, coconut shell, petroleum cock, and petroleum pitch. Activated carbon is widely used for water purification applications. Different micro-organism, organic compounds, viruses, bacteria, cysts, volatile organic chemicals are treated by activated carbon. There are many literatures describing the effective use of activated carbon in contamination treatments.

Activated carbons (AC) remove bacteria from the aqueous medium through attractive Van der Waals forces. The AC can also be electrostatic positively or negatively charged to attract ionically charged endotoxins, cytokins, or bacteria membrane.

The sachet in the preferred embodiment is made of expanded polytetrafluoroethylene (ePTFE) membrane that is sold by (W. L. Gore & Associates, Inc. Newark, Del., USA) with such a porosity that allows plasma, bacteria, viruses, and prion to pass through. Types of bacteria include but not restricted to Streptococcus pneumonia, Streptococcus, Staphylococcus, Staphylococcus aureus, Escherichia coli, Bacillus, Klebsiella, Serratia, Corynebacteria diphtheria, Mycobacterium tuberculosis, and Chiamydia Pneumonia. Types of viruses include but not restricted to Poxvirus-Variola, Parainfluenza, Respiratory Syncytial, Varicellazoster, HIV, HCV, SARS, Adenovirus, CMV, Togavirus, Echovirus, Rhinovirus, and Parovirus.

In another embodiment, the adsorbent media is made of porous polystyrene resin commercially known as Purolite (Bala Cynwyd, Pa., USA) Large family of polystyrene resins with high BET surface area and high porosity. Some of these resins that are used for this application are PAD550, PAD600, and PAD900. Another family of Purolite resins commercially known as Macronet with resins MN200 and MN400. Another family of porous polystyrene resins is Amberlite XAD16, XAD4, XAD1180, XAD1600, XAD16HP (Rohm and Haas Company, Philadelphia, Pa., USA). Another polymeric adsorbent media that could be used in this embodiment is Dowex optipore L493 supplied by Dow Chemical Company. This porous media has a BET surface area of 400 m²/g to 1300 m²/g is capable to adsorb bacteria and viruses in a fluid environment.

In addition to their porosity and large (BET) surface area characteristics, these resins could also have ion exchange characteristics. Ion exchange resins are classified as cation exchangers, that have positively charged mobile ions available for exchange, and anion exchangers, whose exchangeable ions are negatively charged. Both anion and cation resins are produced from the same basic organic polymers. Resins can be broadly classified as strong or weak acid cation exchangers or strong or weak base anion exchangers.

All these polymeric particulate resins come in spherical shape of different diameter ranging between (0.2 mm to 2 mm) which are too large to penetrate through the sachet membrane. Therefore these resin beads are captured inside the sachet and can not intermix with the platelets outside the sachet.

In other embodiment the sachet walls are coated with hydrogel layer that improves the hemocompatibility of the sachet surface. The hydrogels have thrombo-compatibility characteristics that prevent platelets adhesion to the sachet surface. The hydrogel could be made of material such as Poly (Hydroxyethyl Methacrylate), 2-Hydroxyethyl Methacrylate, or Poly-HEMA also known by CAS Number 25249-16-5. Different classifications of hydrogel based on ionic charges such as anionic, cationic, ampholytic, and neutral hydrogel can be used. Different classifications of hydrogel based on structure such as amorphous, semi-crystalline, and hydrogen-bonded hydrogel can be used. The hydrogel also can be made of Poly (Vinyl alcohol), Poly (N-vinyl 2-pyrrolidone), and Poly (ethylene glycol).

Hydrogel coating of the sachet walls allows for the use of a membrane with porosities that are greater than the size of the platelets.

The sachet Microporous membrane material could be any of the following material but not restricted to Polyethersulfone (PES), Polyester, Polysulfone, Polyvinylidene flouride (PVDF), Nylon, Polytetraflourethylene (PTFE), Cellulose acetate, and Polypropylene.

It should be known that this invention is not restricted to platelet storage bag. It also can be used for concentrated red blood cell (concentrated RBC) storage bag, white blood cell (Leukocytes) storage bag, plasma storage bag, whole blood storage bag, or any combination of RBC, platelets, leukocyets, and plasma.

The porosity of the sachet membrane is selected in accordance with the cell size that is prevented from crossing through the membrane. For example leukocytes are spherical with diameter size (10 μm to 12 μm), RBC size is (7 μm Diameter and 2 μm thick), and Platelets are disc shape with (2 μm to 4 μm) in size. The sachet membrane porosity used in the leukocyte storage bag is larger than the membrane porosity used for the RBC storage bag. The sachet membrane porosity used for the RBC storage bag is larger than the membrane porosity used for the platelets storage bag. The porosity of the sachet membrane used for the whole blood should prevent all cells from passing through, therefore the porosity size should selected to prevent the smallest cell which is the platelet from passing through.

The adsorbent media inside the sachet can be selected from the family of porous polymer resins, activated carbon particulates, or activated carbon cloth. The adsorbent media could be any combination of these activated carbon and porous polymer resins.

In some cases the adsorbent media biocompatibility characteristics can be enhanced by coating the surface. One of the coating techniques known in the industry is hydrogel. One of very well known hydrogel is Poly-HEMA, PHEMA, or Poly (2-hydroxyethyl methacrylate). Other kinds of hydrogel such as Polyvinyl Alcohol (PVA), Polyethylene Glycol (PEG), Polyvinyl pyrrolidone, and Ethylene glycol dimethacrylate (EGDMA) can be used. Other coating techniques that could be used to enhance the biocompatibility characteristics of the adsorbent carbon or resins are Cellulose, Silicone, and Poly-Methyl Methacrylate (PMMA).

Based on the foregoing, an object of the present invention is to provide an improved passive storage system for blood products and more particularly for platelet concentrate for removing contaminants and pathogens from the transfused platelets. A specific object includes providing passive storage system comprising activated carbon fibers which removes a broad spectrum of contaminants, including very small microorganisms such as bacteriophage to much larger pathogens such as E. coli bacteria. Furthermore the storage system comprising polymeric porous resins and ion exchange resins for the removal of pathogens from the transfused platelets.

Activated carbon and porous polymeric resins having a tendency to adsorb bacteria from solutions can be employed, thereby minimizing the risk of septicemia acquired in the course of a transfusion.

The removal of such pathogens from platelets concentrate using the present passive storage without any chemical additive is at a level not previously demonstrated by the prior art. Another object of the present invention is to provide a method of removing pathogens from blood products, particularly concentrated platelet, using the storage container of the present invention.

Another object of the invention is to provide an article of manufacture comprising the storage container of the present invention.

Another object of the present invention is that the in vivo shelf life of blood platelets can be extended beyond those currently attainable in the prior art by providing adsorbing activated carbon capable of supporting platelet metabolism.

Another object of the present invention is that the in vivo shelf life of blood platelets can be extended beyond those currently attainable in the prior art by providing adsorbing and ionic charged resin capable of supporting platelet metabolism.

Another object of the present invention is to provide a platelet storage system which promotes the preservation of the platelet morphology.

Another object of the present invention is to provide a platelet storage system which buffers the pH of stored platelets.

Another object of the present invention is to provide a platelet storage system which extends the functional life of platelets.

In a different embodiment of the invention, the blood or the blood components are not confined inside a container but rather they are flowing through a system of connecting tubes and containers. For example in apheresis system blood flows from a donor in a closed extracorporeal circuit to be processed by the system and then the blood or certain components of the blood return back to the donor. In this embodiment the blood flows through a mass of activated carbon or porous resin with or without ion exchange characteristics. As it is explained above in this study, the activated carbon and the porous resin capture bacteria, cytokines, endotoxins, and other micro-organisms and toxins that are suspended in the blood. When blood passes through a mass of ionic exchange resin, different types of ions suspended in the blood are captured by the resin.

More specifically, when blood is drawn from an apheresis donor it is mixed with anticoagulant in order to avoid clotting. Depending on the type of the anticoagulant there is a defined ratio for mixing the anticoagulant with blood. The mixture of blood and anticoagulant is called anticoagulated blood. After processing of the anticoagulated blood by the apheresis system, some blood components (Plasma, RBC, or Platelets) are stored in containers for future reinfusion. The rest of the blood components are returned to the donor. It is typical in apheresis procedure to process large amount of donor's blood (in the range of 5 to 6 liters) that flows continuously or in batches (depending on the Apheresis system) from the donor to the system and back again to the donor. The blood flows at a rate that is comfortable to the donor (in a range of 50 to 150 ml/minute) without violating the limit of the allowed extracorporeal volume of the blood. As the blood exits the vein of the donor it is immediately mixed with anticoagulant at a constant ratio. For example for apheresis the ratio is (1:16). As the blood is being processed, it is constantly mixed with anticoagulant. When the blood is returned back to the donor, a large amount of anticoagulant is infused into the donor. For an apheresis procedure that processes 5 liters of blood, more than 300 ml of anticoagulant with citrate content of (0.3 to 0.4 g/100 ml) is infused into the donor. In most cases this amount of citrated anticoagulant causes discomfort to the donor, especially after long apheresis procedures. Approved anticoagulant-preservatives include acid-citrate-dextrose solution (ACD), citrate-phosphate-dextrose solution (CPD), citrate-phosphate-dextrose-dextrose solution (CP2D), and citrate-phosphate-dextrose-adenine solution (CPDA-1). Removing the anticoagulant from the blood that is returned to the donors would increase their level of comfort. Directing the returned blood to pass through a chamber containing ionic exchange resins would help in removing citrate from the blood before it is infused into the donor. More specifically a chamber containing anion exchange resin can remove citrate from the blood. Anion exchange resins, i.e., those possessing functional groups which can undergo reactions with anions in a surrounding solution, particularly weakly basic anion exchange resins, are preferred. Such resins are formed of Styrenic or Acrylic porous matrix with high mechanical stability. Such resins used in the present invention with apheresis systems have the additional properties of adsorbing acids from organic reaction mixtures, exchanging anions in a slightly acidic media, a high exchange capacity, low swelling properties and a tendency to adsorb bacteria from the surrounding solution are particularly advantageous. The anion exchange resin may be used alone or in combination with other anion and/or cation exchange resins suitable for the intended purpose. Weak Base Anion Exchanger resins are commercially available under the trade name of Amberlite IRA92 or IRA96 from Rohm & Haas Company, Macroporous Polystyrenic Purolite A100 and A835 from Purolite, and Dowex MWA-1 or Dowex M43 from Dow Chemical.

In another embodiment of the present invention a strong base anion exchanger resins such as Purolite A500 and A510 from Purolite are used to extract acidic solution such as citrate from the returned blood.

In another embodiment of the present invention activated carbon is used to extract acidic solution such as citrate from the returned blood.

In another embodiment of the present invention different combinations of activated carbon, weak base anion resin, and strong base anion resin are used to extract acidic solution such as citrate from the returned blood.

In another embodiment adsorbent porous resins with high BET surface area and high porosity such as PAD400 and PAD600 from Purolite, Amberlite XAD4 and XAD16 from Rohm and Haas, and Dowex L493 from Dow Chemical are used in the housing that is positioned on the blood return line. As the blood components are re-infused back into the donor, they are directed to pass through the housing containing the adsorbent porous resins. In most apheresis systems, as the blood is drawn from the donor, processed and returned back to the donor; few red blood cells (RBC) are hemolized (damaged) due to different stresses during the processing steps.

Hemolized RBC release hemoglobin to the plasma medium. The free hemoglobin could negatively affect the donor and especially damaging to the kidneys as it is re-infused back with the returned blood components. The adsorbent porous resins have the capacity to adsorb the free hemoglobin from the returned blood components flow. Therefore preventing the free hemoglobin from being re-infused back into the donor.

In another embodiment activated carbon cloth or activated carbon particulates are used in the housing that is positioned on the return line to adsorb the free hemoglobin from the returned blood products before they are re-infused back into the donor. The activated carbon can be used in this housing with or without the adsorbent porous resins.

Citrate is used as anticoagulation in apheresis or dialysis applications. When blood is drawn from a subject and mixed with anticoagulant to be cycled in an extracorporeal circuit repeatedly, the anticoagulant concentration increases in the body of the subject. In platelet pheresis the mean plasma citrate concentration in the donor progressively increase during the apheresis application and reach up to a median of 1.6×10⁻³ Mole (1.6 mM). This leads to a decrease in ionized calcium. The ionized calcium level drop from the base line can reach 33% on average. This can lead to symptoms of citrate toxicity such as paresthesias (predominantly perioral and acral), light-headedness, tremor and shivering. Hypocalcaemia may cause vascular smooth muscle relaxation, depressed myocardial function, arrhythmia and chronic metabolic (late) effects of citrate (e.g. bone demineralization). Prophylactic oral calcium was associated with only modest improvements in these citrate-induced symptoms.

Citrate also binds magnesium. Citrate administration during dialysis or apheresis applications decrease ionized magnesium by about 30%. This can cause symptoms such as muscle weakness, muscle spasms and impaired myocardial contractility. Steady-state plasma ionized calcium levels inversely correlate with the QT interval. In cardiology, the QT interval is a measure of the time between the start of the Q wave and the end of the T wave in the heart's electrical cycle. Prolongation of the QTc interval during plateletpheresis is a general finding. It can be considered as a sensitive marker of citrate toxicity at the myocardial tissue level. Citrate infusion has been associated with drop in blood pressure, decreased cardiac output and cardiac arrest.

Patients subjected to hemodialysis and hemofiltartion processes where their blood is circulated in an extracorporeal circuit are induced with citrate that is used to anticoagulate the blood during dialysis operation. Platelets donors and two red blood cell units (2 RBC units) donors are induced with citrate that is used to anticoagulate the blood during apheresis application. Dialysis patients and repeated apheresis donors are exposed to citrate that discomfort them and expose them to long term cardiology problems. Having a device and a method to extract the citrate from the anticoagulated blood just before it is reinfused into the patient's or the donor's body. The citrate filter (anticoagulant filter) is positioned downstream of the extracorporeal circuit (EC) and very close to the reinfusion site. This is done to ensure that the blood is perfectly anticoagulated while it circulates in the EC. The citrate is extracted by the anticoagulant filter (citrate filter) at the vicinity of the reinfusion site to minimize the path of the anticoagulant free blood flow. A filter screen can be added between the anticoagulant filter and the reinfusion site for safety purposes to prevent any inadvertent clot or any loose filtration (ion exchange resin) particulates from being re-infused.

It should be noted that the extracorporeal circuit for the dialysis system or for any apheresis system (2 RBC units or Platelets) can be modified to have a bypass channel to direct the blood flow around the anticoagulant filter. This is important especially for the dialysis or the apheresis systems that utilize the batch (non-continuous) processing, and also for the systems that utilize the same venipuncture site for blood draw and reinfusion. In this case the majority of the blood (or blood products) returning to the human subject is directed through the anticoagulant filter (citrate filter) to extract the citrate, the last chunk (small volume) of the blood is directed to bypass the filter. This is done in order to keep the section of the extracorporeal circuit between the anticoagulant filter and the venipuncture site; filled with coagulated blood toward the end of the batching procedure to prevent any clotting in case the blood (or blood products) flow is stopped. In other cases, the section of the extracorporeal circuit between the anticoagulant filter and the venipuncture site; is flushed with saline (or other therapeutic solution). This is done toward the end of the batching procedure to prevent any clotting in case the blood (or blood products) flow is stopped. In this case, the circuit section post anticoagulant filter is filled with saline when the batch procedure is stopped. Therefore, no clotting can occur in the post anticoagulant filter section.

Further aspects, features and advantages of the present invention will become more fully apparent from the following description of specific embodiments, the attached drawings and the appended claims; to those skilled in the art to which this invention pertains.

### BRIEF DESCRIPTION OF THE DRAWINGS

The foregoing and other objects, features and advantages of the invention will be apparent from the following more particular description of preferred embodiments of the drawings in which like reference characters refer to the same parts throughout the different views. The drawings are not necessarily to scale, emphasis instead being placed upon illustrating the principles of the invention.

FIG. 1A—A schematic top view of the storage container demonstrating the activated carbon cloth enveloped inside a porous sachet.

FIG. 1B—A schematic top view of the storage container demonstrating the activated carbon cloth enveloped inside a porous sachet having structural beams.

FIG. 2A—A schematic side view of the storage container demonstrating the activated carbon cloth enveloped inside a porous sachet.

FIG. 2B—A schematic side view of the storage container demonstrating the activated carbon cloth enveloped inside a porous sachet having structural beams.

FIG. 3—A schematic side view of the storage container demonstrating the activated media particulates inside meshed pockets that are attached to the activated carbon cloth with the whole assembly enveloped inside a porous sachet.

FIG. 4—A schematic side view of the storage container demonstrating the activated media particulates stored inside an activated carbon cloth packet that is enveloped inside a porous sachet.

FIG. 5—A schematic side view of the storage container demonstrating the activated media particulates inside a meshed packet that lies besides an activated carbon cloth with the whole assembly is covered by a porous sachet.

FIG. 6—A schematic side view of the storage container demonstrating activated media particulates inside a meshed packet that is covered by a porous sachet.

FIG. 7—A schematic view of the blood circuit between a donor and apheresis system (two needles access) demonstrating the resin chamber on the blood return line.

FIG. 8—A schematic view of the blood circuit between a donor and apheresis system (one needle access) demonstrating the resin chamber on the blood return line.

FIG. 9—A schematic view of the resin chamber with a pouch containing adsorbent resin.

FIG. 10—A schematic view of the resin chamber with screens.

FIG. 11—Schematic view of extracorporeal circuit with free hemoglobin filter.

FIG. 12—Free hemoglobin/Prion filter with a pouch containing adsorbent resin.

FIG. 13—Free hemoglobin/Prion filter with screens.

FIG. 14—Free hemoglobin/Prion filter with structured adsorbent media.

FIG. 15—Blood Reservoir with adsorbent capacity.

FIG. 16—Platelet storage container having loose or pouched adsorbent media.

FIG. 17—Platelet storage container built with compartments encapsulating adsorbent media or activated carbon.

FIG. 18—Platelet storage container having at least one zone containing adsorbent media separated by porous membrane from a zone that contain platelets.

FIGS. 19A, 19B, 19C, & 19D—Platelet storage container having a laminated outer-wall that has adsorbent media layer and porous membrane layer.

FIG. 20—Schematic view of extracorporeal circuit with anticoagulant filter bypass.

FIGS. 21A & 21B—Platelet storage container with integrated tubing set.

### DETAILED DESCRIPTION OF THE INVENTION

It should be apparent to those skilled in the art to which the present invention pertains that a number of techniques can be employed to provide means that improves platelet concentrate storage conditions by maintaining the level of the pH in the solution, and reducing the pathogen count in the transfused product. These improving means can also apply to other blood products.

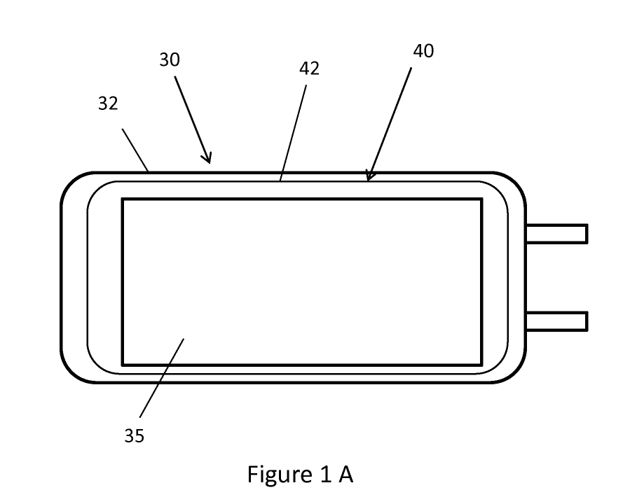

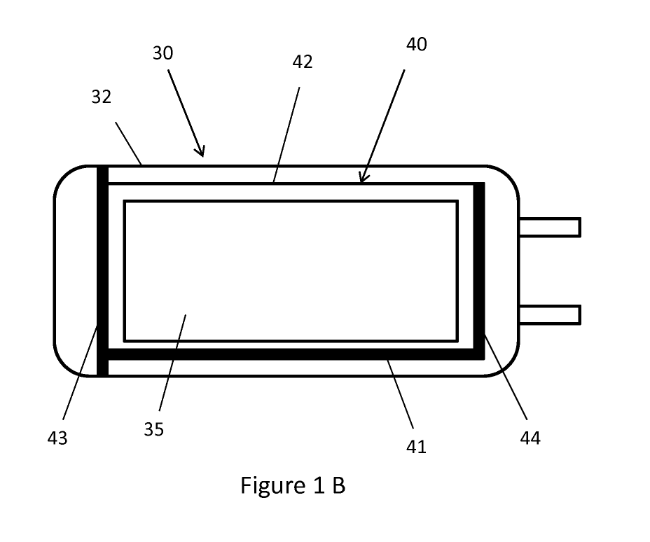

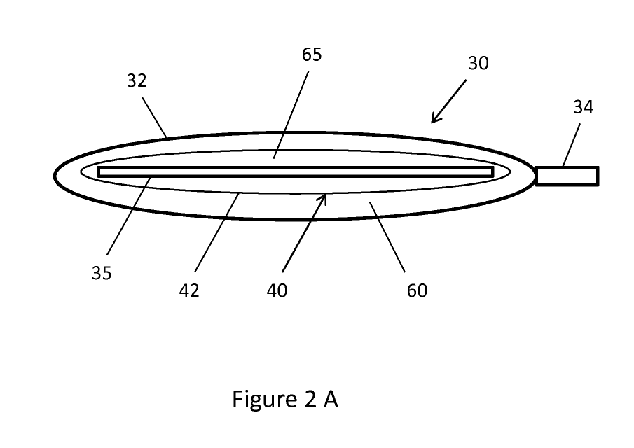

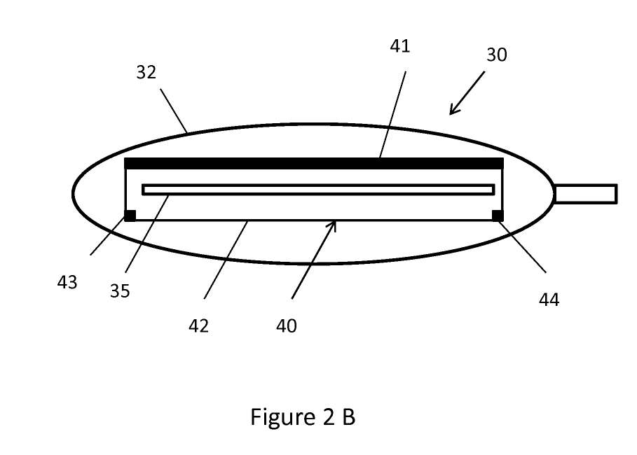

Referring to FIG. 1A, a top view of the storage container, and FIG. 2A, a cross sectional view of the container. The storage container 30 is typically made of welded plastic sheets that form the outer wall 32. A port 34 is used to communicate fluids between the inner and the outer parts of the container. The adsorbent cloth 35 extends longitudinally inside the container. In the preferred embodiment the adsorbent cloth is activated carbon cloth. The adsorbent cloth is completely enveloped by a sachet 40 that it is made of porous membrane 42. This membrane has a porosity that allows bacteria and viruses suspended in the plasma to pass through with the plasma. The membrane porosity is small enough to prevent any access of platelets, leukocytes, and RBC through the membrane. As it is depicted in FIG. 2, the porous membrane 42 divides the space inside the storage container in to two separate zones. The first zone 60 is defined by the space occupied between the inner surface of wall 32 and the outer surface of the sachet 40. The second zone 65 is defined by the space occupied inside the sachet 40.

FIG. 1B, a top view of the storage container 30 show the sachet 40 having a defined structure. FIG. 2B show a cross sectional view of the same container with the structures sachet. The sachet could have a parallelepiped or a cylindrical structure. The structure could be supported by rigid or semi rigid lateral or longitudinal beams. Lateral beam 44 support the sachet but are not connected to the container wall 32, while lateral beam 43 connect to the outer wall and hold the sachet fixedly in place. The longitudinal beam 41 extends along the sachet and could cover the whole length of the sachet or a partial section of it. This longitudinal beam could remain independent of the outer wall or could be connected to it. The structured shape sachet is used to facilitate the flow of the plasma or other fluids in and out of the sachet, and therefore facilitate the flow of the pathogens inside the sachet to come in contact with the adsorbent media 35. The structure beams of the sachet are connected to the outer wall of the container in order to prevent any movement of the sachet with respect to the container. Even when the container is placed on a shaker. Preventing the movement of the sachet with respect to the container will help in preventing or reducing platelet activation.

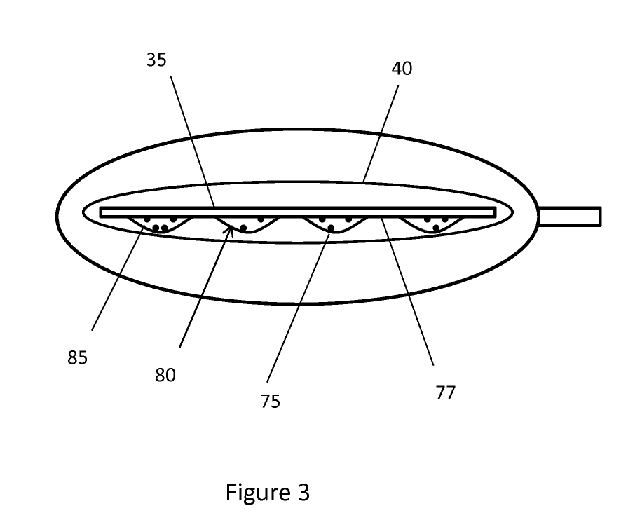

In blood banks, platelet rich plasma or concentrated platelet solution are introduced in the storage container that is placed on a shaking platform at 22° C. The storage container 30 in the current invention receives the concentrated platelet solution in a normal way and the container is placed on a shaker with a constant horizontal agitation at approximately 70 to 80 cycles per minute and at 22° C. The concentrated platelet solution that is mainly a mixture of platelets and plasma move inside the container due to the platform movement. This movement generates plasma flow between the two zones in and out of the sachet through the porous membrane 42. Plasma can flow through the porous membrane. Bacteria, viruses, cytokines, toxins, endotoxins, and other micro-organisms that are suspended in the plasma, flow through the membrane as well. The porosity size of the membrane allow for these pathogens to pass through and enter in to the sachet. The Platelets have a larger size of the membrane pores therefore; they cannot enter inside the sachet. The pathogens that enter the sachet come in contact with the adsorbent media which is made of activated carbon cloth. The adsorbent media is made of activated carbon fibers that have very large surface area. The pathogens stick to the surface of the adsorbent media and become captured inside the sachet. Platelets and other blood cells inside the storage container, remain in the area outside the sachet. As the pathogens enter the sachet and become trapped inside, they are readily separated from the platelet concentrate. Therefore the number of the pathogens in the platelet concentrate drop. When the concentrated platelets solution is used for transfusion, it has a lower population of pathogens. In another embodiment of the invention, other adsorptive particulates are added to the activated carbon cloth to increase the adsorption capacity of the media inside the sachet. Referring to FIG. 3 adsorbent particulates 75 are captured in special pockets 80 that are formed by joining a meshed sheet 85 to the adsorbent cloth 35 at spots 77. The mesh could have a porosity ranging from 50 μm to 300 μm depending on adsorbent particulate size. The mesh porosity is to allow for the plasma to flow freely in to the pocket containing the adsorbent without having the adsorbing particulates exiting the pocket. Typically in most cases the mesh size is 170 μm. Furthermore, these particulates 75 could be mad of activated carbon having granular, pallet, or spherical shapes. These particulates could also be made of porous polymeric material or resins having different shapes and most likely spherical shape. These particulate resins can also be characterized as ion exchange resin. These resins could be of the family of Purolite, Amberlite, Optipore, or Dowex. In different embodiment of this invention, pockets 80 could be formed by joining meshed sheet 85 to both sides of the adsorbent cloth 35.

For use in the platelet storage system of the present invention, anion exchange resins, and more particularly weakly basic anion exchange resins, are preferred. These weakly basic resin exhibit minimum exchange capacity above a pH of 7.0 and are good to experience reactions with anions where they only adsorb acids from the surrounding medium. Such resins which have the additional properties of adsorbing acids from organic reaction mixtures, exchanging anions in a slightly acidic media, a high exchange capacity, low swelling properties and a tendency to adsorb bacteria from the surrounding solution are particularly advantageous. Glucose in the platelet concentrate solution starts to metabolize. In case of insufficient oxygen supply to the storage bag, glucose is metabolized anaerobically resulting in the production of lactic acid. The excess generation of this lactic acid causes a drop in the stored solution pH. If the pH level drops from 6.8 to 6.0, the platelets progressively change shape from discs to spheres. If the pH falls below 6.0, then platelets become nonviable after infusion in vivo. The presence of the anionic exchange resin in the storage bag and its capability in adsorbing the lactic acid from the medium, it neutralizes the pH in the solution and establishes a favorable environment for the platelets. Therefore the platelets remain viable and effective for post storage in vivo infusion. Such resins are commercially available under the trade name of Amberlite from Rohm & Haas Company, weakly basic, polystyrene-polyamine type anion exchange resin having a styrene-divinylbenzene matrix. Other commercially available ion exchange resins are Purosorb and Macronet from Purolite and Dowex from Dow Chemical.

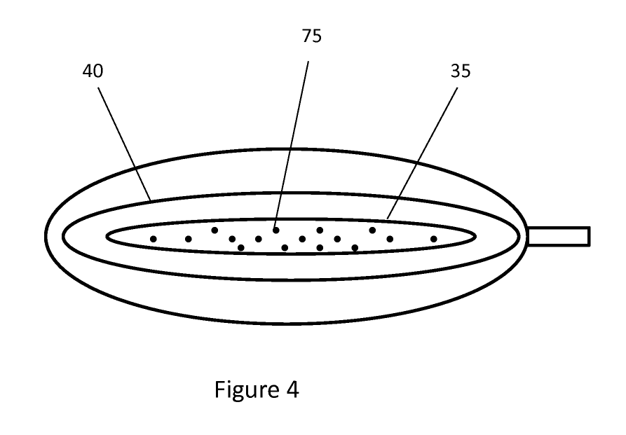

In another embodiment of the invention, adsorbent particulates are amassed in a pouch made of the activated carbon cloth that is inserted in a sachet inside the storage container. Referring to FIG. 4 adsorbent particulates 75 are stored inside a pouch 90 that is made of the same materials as the adsorbent cloth 35. The whole pouch including the adsorbent particulates is placed inside a sachet 40 that is made of a porous membrane 42. The sachet is situated inside the concentrated platelets storage bag where the plasma is free to flow in and out of the sachet. It is clear that the porosity of the sachet membrane does not allow the platelets or any blood cell to pass through.

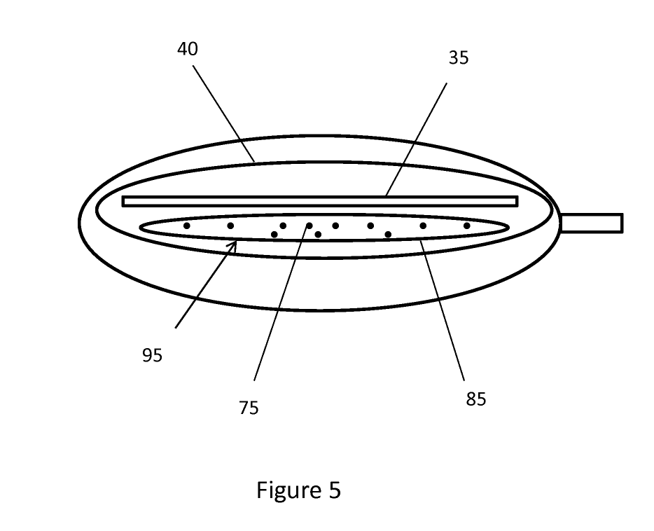

FIG. 5 demonstrates another embodiment where the adsorbent particulate 75 are stored in a pouch 95 made of the meshed sheet 87 with a porosity ranging between 50μ to 300μ as the same sheet material used in the embodiment of FIG. 3. In this particular embodiment the pouch 95 extends longitudinally along the storage container the same as the adsorbent cloth 35. A sachet 40 includes both of the pouch 95 and the adsorbent cloth 35 and it is placed inside the storage container.

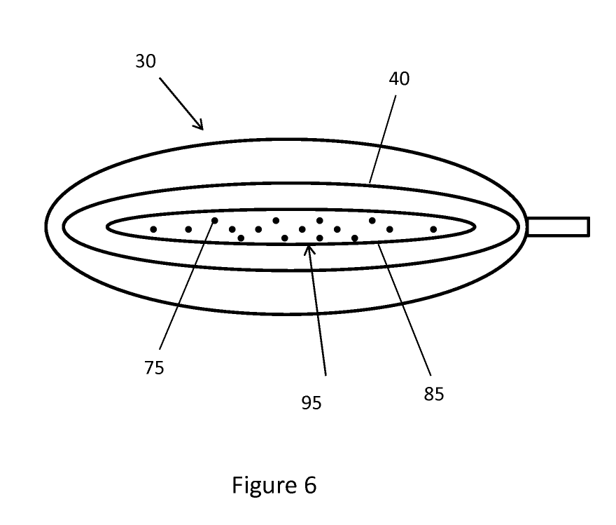

In another embodiment demonstrated in FIG. 6 the pouch 95 encompassing adsorbent particulates 75, is placed inside the sachet 40 that extends within the storage container. The pouch 95 is built with meshed material with porosity ranging between 50 μm to 300 μm. The sachet is built with a membrane having a porosity that allows the plasma to pass through but preventing the platelets. Multiple packets can be used in the same storage container. Different packets can have different types of adsorptive particulates. For example one packet can have activated carbon spheres or granules and another packet can have polymeric resin beads or ionic exchange beads.

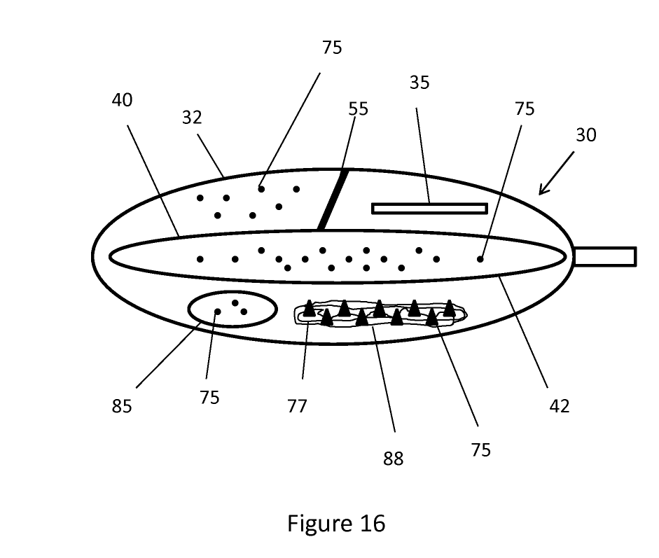

In a new embodiment of the platelet storage container 30 is depicted in FIG. 16. The container is built with outer layer 32 that is permeable to gases such as Oxygen (O₂) and Carbon dioxide (CO₂) and it is made of material that can be sterilized. The outer layer 32 can be made of flexible, semi flexible, or rigid material. The storage container 30 can have flexible, semi flexible, or rigid structure. The storage container is designed to hold blood, blood components, platelets concentrate, red blood cell concentrate, and plasma. The container has a pouch 40 that is made of porous membrane 42. The porous membrane allows the plasma fluid and the pathogens to pass through while preventing platelets and other blood cells from penetrating through. The porous membrane is preferably hydrophilic to facilitate the penetration of the fluids. The pouch 40 encompasses adsorbent media 75 that could be activated carbon, styrene resin, Acrylic resins, ion exchange resins, or other porous resin. The adsorbent media could be coated with hydrogel polymers or by other resin polymers for biocompatibility purposes. Therefore when platelet concentrate is stored in the container 30, the plasma penetrates through the membrane 42 entering the pouch 40 while the platelets are remained outside. The container is placed on an oscillating shaker and the plasma flows in and out of the pouch by the shaking movement. As the plasma flows inside the pouch, bacteria or pathogens with a size of about 1 micron (μm) or less flows with the plasma inside the pouch to come in direct contact with the adsorbent. The pathogens stick to the adsorbent and are trapped on its surface until it die. This phenomenon of killing and sticking of pathogen to the adsorbent continuously take place from the moment that the platelet rich plasma (platelet concentrate) is introduced in to the container until it is taken out of the container. While the platelet concentrate is residing inside the container, the plasma flows through the membrane 42 and the pathogens flow with the plasma through the membrane to come in contact with the adsorbent media. The adsorbent media will actively and continuously adsorb and kill the pathogens throughout the whole storage period. The movement of the platform facilitates the flow of plasma and the pathogens through the membrane 42 and makes it easier to absorb the pathogens. The continuous killing of the pathogens can easily extend the platelet storage time (7 days or more) while maintaining platelets safety and quality. Some of the adsorbents are impregnated with noble metals to kill the pathogens. For example, activated carbon particulates are impregnated with silver or Zinc to increase its capabilities in killing the bacteria that adhere to its surface. The adsorbents are loosely amassed inside the pouch or they are structured in a matrix 88. The adsorbent media 75 are captured in resin polymers 77 to form an adsorbent matrix 88. The term Pathogens refer to infectious agent or microorganism (in the widest sense) such as a virus, bacterium, prion, or fungus that causes disease in its host. The host may be an animal (including humans), a plant, or even another microorganism. Types of bacteria include but not restricted to Streptococcus pneumonia, Streptococcus, Staphylococcus, Staphylococcus aureus, Staphylococcus Epidermidis, Staphylococcus Agalactiae, Escherichia coli, Bacillus, Bacillus Cereus, Clostridium Perfringens, Enterobacter Aerogenes, Klebsiella, Klebsiella Pneumonia, Serratia, Serratia Marcescens, Corynebacteria diphtheria, Pseudomonas Aeruginosa, Mycobacterium tuberculosis, and Chiamydia Pneumonia. Types of viruses include but not restricted to Poxvirus-Variola, Parainfluenza, Respiratory Syncytial, Varicellazoster, HIV, HCV, SARS, Adenovirus, CMV, Togavirus, Echovirus, Rhinovirus, and Parovirus.

The micro-porous membrane 42 material could be any of the following material but not restricted to Polyethersulfone (PES), Polyester, Polysulfone, Polyvinylidene flouride (PVDF), Nylon, Polytetraflourethylene (PTFE), Cellulose acetate, and Polypropylene. It is preferred to have the membrane 42 made of material that is biocompatible to prevent thrombosis and greatly minimizes platelet activation and platelet adhesion to its surface. In some cases the micro-porous membrane 42 can be made of the same material of the outer wall 32. This membrane could have large number of small pores of size (1 μm or 2 μm) that are drilled through the membrane. Some pore holes could be drilled by laser beams. The micro-porous membrane 42 could be coated with hydrogel layer that improves the hemocompatibility of the surface. The hydrogels have thrombo-compatibility characteristics that prevent platelets activation and platelets adhesion to the sachet surface. The membrane 42 can be coated with silicone or cellulose to improve the biocompatibility characteristics of its surface. It is recommended to maintain the surface area of the membrane 42 to the minimum possible area to reduce the effect of thrombosis, platelets activation, and platelets adhesion.

The porosities of membrane 42 are selected to prevent platelets from passing through and entering inside the pouch or the zone where the adsorbent media is located. Similarly, the small pores of membrane 42 prevent any particulates of the adsorbent media that have the size equal to or larger than the platelets from mixing with the stored platelet solution. Adsorbent media particulate that is capable to pass through the porous membrane to mix with the platelet solution must be much smaller than the platelets. In case that the membrane porosity was selected to be (1 μm) then the size of the adsorbent particulate that passes through the membrane must be less than (1 μm). In general the size of the adsorbent particulates that pass through the membrane porosities must be smaller than the platelets. When these adsorbent particulates are floating in the platelet solution inside the storage container, they could come in contact with larger size platelets. Adsorbent media such as activated carbon or porous resin has light density generally about (0.55 g/ml+/−0.15 g/ml). In case of activated carbon the density can be as low as 0.15 g/ml. The platelets have the density about (1 g/ml). The adsorbent particulate that passes through the membrane would have a size smaller than the platelets and a density about half of the density of the platelets. These adsorbent particulate could not provide a large enough surface for the platelets to adhere to. Also, the mass of each of these particulates is less than the mass of a single platelet cell. The low density adsorbent particulates float on the surface of the fluid inside that container while the platelets are suspended in the fluid. This phenomenon contributes in avoiding contacts between adsorbent particulates and platelets, therefore minimizing platelets activation. It is recommended to wash the adsorbent media thoroughly before it is assembled in platelet storage container. Ultrasonic cleaning can be used to eliminate all small particles and dust residue generated from the adsorbent media. Cleaning can take place for the adsorbent media by itself first, and then another cleaning step can take place after the media is sealed inside the sachet. In this last step the whole sachet with the media inside it are cleaned by ultrasonic cleaner.

The adsorbent media and particularly the activated carbon media is thoroughly cleaned before it is built in the storage container. These media are cleaned in order to eliminate any dust or particulates that could break free and slip through the porous membrane 42 to come in contact with the platelets or other blood cells. The adsorbent media and the activated carbon media are ultrasonically cleaned in de-ionized solution, Ethanol, or Alcohol solutions. The edge of the activated carbon cloth 35 are sealed by Hydrogel, Silicone, PMMA, or Paraffin wax in order to prevent any dust generation or breaking away of any particulate from the edge where the cloth is cut. The edge sealing concept is used to protect the carbon matrix structure.

The pouch 40 containing the adsorbent media can be welded to the container wall 32. A weld 55 is used to fixate the pouch 40 inside the container 30 in order to prevent it from moving loosely while the container is agitated or placed on a moving platform, as shown in FIG. 16. The pouch can be fixated to prevent platelets activation.

The platelet container of the current invention was tested for bacterial contamination with respect to standard platelet bags used in the industry; at Bioscience Research Associates, Inc (BSR), 767C Concord Avenue, Cambridge, Mass. 02138, USA. The testing design included two containers of the current invention, one standard platelet bag for positive control, and one standard platelet bag for negative control. All containers were filled by 80 ml of human platelet concentrate. Two platelet containers of the current invention with a standard platelet bag used as a positive control were induced by 120 CFU/ml of Staphylococcus Aureus bacteria (CFU: Colony-Forming Unit). All bags and containers were placed on shakers (60 to 80 cycles per minute) at room temperature (22° C.). Bacteria culture test were conducted on samples taken from these bags and containers after 5 days and 7 days of storage. (See Table 1)

TABLE 1
Colony-Forming Unit (CFU)

| | After | |
|---|---|---|
| | 5 Days storage | After 7 Days Storage |
| | Concentration | Concentration |
| Samples | (CFU/ml) | (CFU/ml) |
| Negative Control | 0 | 0 |
| Positive Control | 1.7 × 10⁷ | 1.7 × 10⁷ |
| Current Invention Container 1 | 0 | 2,920 |
| Current Invention Container 2 | 0 | 0 |

These results indicate that the current invention container can completely eliminate Staphylococcus Aureus bacteria from platelet concentrate after 5 days storage. The Staphylococcus Aureus bacteria are the most common bacteria that are found in blood products. These bacteria are originated from the skin surface of the donor and are transmitted to the blood bag by the needle venipuncture. Bacteria count on day 7 for the current invention platelet container is very minimal. One container has zero (0) bacteria and the second container has less than 3,000 CFU/ml. The threshold detection limit for the Verax Biomedical Inc. (Worcester, Mass., USA) testing kit for the Staphylococcus Aureus bacteria is (8.3×10³ CFU/ml) after 5 days storage. The Verax PGD testing kit was approved by the FDA for testing for bacteria presence in platelet concentrate product stored for up to 5 days at 22° C. The bacteria counts in platelet concentrate stored for seven days in the current invention containers are well below the threshold limit for Verax system that was approved by the FDA after 5 days storage

In another embodiment of the current invention of the storage container is that adsorbent matrix 88 is placed inside the container 32 without having a membrane 40 to prevent the blood cells from contacting the adsorbent media as shown in FIG. 16. The adsorbents could be coated with hydrogel or other polymers for biocompatibility purposes.

In another embodiment of the current invention of the storage container is that adsorbent media 75 are placed loosely inside the container 32 without having a membrane 40 to prevent the blood cells from contacting the adsorbent media as shown in FIG. 16. The adsorbents could be coated with hydrogel or other polymers for biocompatibility purposes.

In another embodiment of the current invention of the storage container is that adsorbent media 75 are packed inside a pouch made of membrane 85 with porosity large enough to allow all blood cells to pass through but preventing any media particulate from exiting the pouch as shown in FIG. 16. The pouch that is made of membrane 85 is placed inside the container 32 and all the blood cells can come in contact with the adsorbent media. The adsorbents could be coated with hydrogel or other polymers for biocompatibility purposes. The adsorbent media 75 could be made of activated carbon micro-fibers, nano fibers, or nano tubes with high porosity and large surface area.

In another embodiment of the current invention of the storage container is that the adsorbent carbon cloths 35 are placed loosely inside the container 32 without having a membrane 40 to prevent the blood cells from contacting the adsorbent media as shown in FIG. 16. The carbon cloths could be coated with hydrogel or other polymers for biocompatibility purposes. It should be noted that the biocompatible pouch that is used to prevent the blood cells from touching the adsorbent media; can be fabricated by using different membranes of different porosities. Each membrane layer can independently cover a section of the pouch. These membrane materials can have different porosities or no porosity at all.

The pouch can also be made of multi-layered membrane of different materials with different porosities. Each layer of the pouch can be made of sections of different membrane with different porosities. Some sections of the pouch could be made of biocompatibility materials with no porosity.

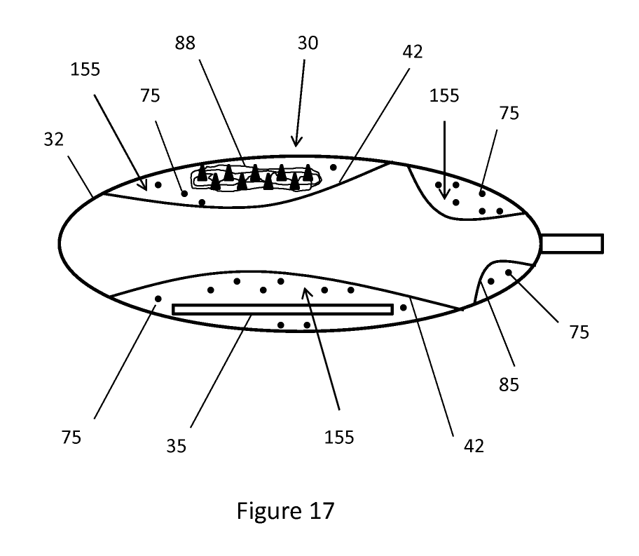

Referring to FIG. 17, another embodiment of the current invention is demonstrated by having the adsorbent media including activated carbon encapsulated in one or more compartments confined between the outer layer of the storage container and the protective porous membrane inside platelet storage container. These compartments (zones) extend from the outer wall 32 of the storage container. FIG. 17 depicts multiple compartments 155 (or zones) for encapsulating different forms of adsorbents between the outer wall 32 of the container and porous membrane 42 or porous membrane 85. Different compartments 155 are demonstrated in FIG. 17 extend from the container outer wall 32 and have porous walls that are in direct contact with the stored fluid. These compartments are completely contained inside the storage container and can be of any size or shape. The compartment can have at least two walls, one is the outer wall 32 and the other is a porous membrane wall 42. The compartment can have the outer wall 32 as one of its own walls. It is noted that any compartment (zone) can be used independently in the storage container. The storage container can be built with one type of the illustrated compartments (zones) or it can be built with a combination of different zones. In one compartment 155 activated carbon cloth 35 is sandwiched inside the blood cell storage container between the wall 32 and a porous membrane 42. When blood cells mixed with plasma are stored in the container, the plasma can be flushed through the membrane to become in direct contact with the carbon cloth while all the cells remain out of the compartment (zone) containing the carbon cloth. The porosity of the membrane can be selected to keep the platelets or the red cell out of the zone containing the adsorbent media while allowing for the pathogens to flow through the membrane with the plasma fluid. The porosity of the membrane can be (1 μm to less than 2 μm) to prevent platelets (2 μm to 4 μm in size) from penetrating through the membrane while the pathogens (less than 1 μm) can pass through. Membrane with porosity of (5 μm) allows for the platelets and the pathogens to pass through while preventing the red cells (7 μm) from passing through. Membranes with porosity of (15 μm) allow all blood components to pass through. The maximum porosity of the membrane can be defined in a way to keep the adsorbent media inside the zone or inside the pouch. The maximum porosity has to be less than the minimum size of the adsorbent media. The membrane material has to be biocompatible and it is made of material that can be sterilized. The membrane material should be safe to blood components such as preventing platelets from adhering to its surface and do not activate the stored platelets. It is preferred that membrane material is hydrophilic. The same can be said about the container wall 32. The wall material has to be biocompatible, hemocompatible, permeable to O₂ and CO₂, prevent platelet from adhering to its surface, do not activate platelets, do not hemolize red cell, and it is made of material that can be sterilized. Commercial examples of the container wall 32 include Baxter PL-732, Haemonetics CLX, Gambro Citrate PVC, and Baxter PL-3014.

Referring to FIG. 17, the adsorbent media can be activated carbon cloth 35, activated carbon matrix 88, or loose adsorbent particulates 75. The adsorbent particulates include activated carbon, porous styrene resin, porous acrylic resin, and other porous resin. The adsorbent media 75 could be made of activated carbon micro-fibers, nano fibaers, or nano tubes with high porosity and large surface area. The activated carbon can be impregnated with Silver or coated with Silver Nitrate to increase its antibacterial capabilities. All the adsorbent media can be coated with biocompatible and blood safe polymer such as hydrogel, silicon, PMMA, or Cellulose. The hydrogel polymer includes poly-HEMA, PEG, PVP, and PVA polymers. The activated carbon 35 can be cloth, yarn, felt, paper, veil mat, felt rigid, or chop that could be supplied by “KRECA” Kureha America LLC (420 Lexington Ave. Suite 2510, New York, N.Y. 10170, USA). The activated carbon 35 can be woven or knitted cloth made of activated carbon fiber, micro fiber, nano fiber, or nano tube. The activated carbon 35 can be or compressed into a felt pad made of activated carbon fiber, micro fiber, nano fiber, or nano tube. Platelet Additive Solution (PSA) can be added to the container. Commercial examples of the PSA include “InterSol” solution supplied by Fenwal (Lake Zurich, Ill., USA). Other types of platelet additive solutions PSA known in the blood bank industry are PSA-II (T-Sol, SSP), PSA-III (InterSol), PSA-IIIM (SSP+), CompoSol, and M-Sol. In some other applications additive solutions such as AS-1 (Adsol), AS-3 (Nutricel), or AS-5 (Optisol) can be added to the container to nutrition the stored cells. These solutions contain different concentrations of Dextrose, Adenine, Sodium Phosphate, Mannitol, Sodium Chloride, Sodium Citrate, and Citric Acid. In some other applications glucose, lactose, citrate, sodium chloride, sodium citrate, phosphoric acid, citric acid, or adenine can be added to the container.

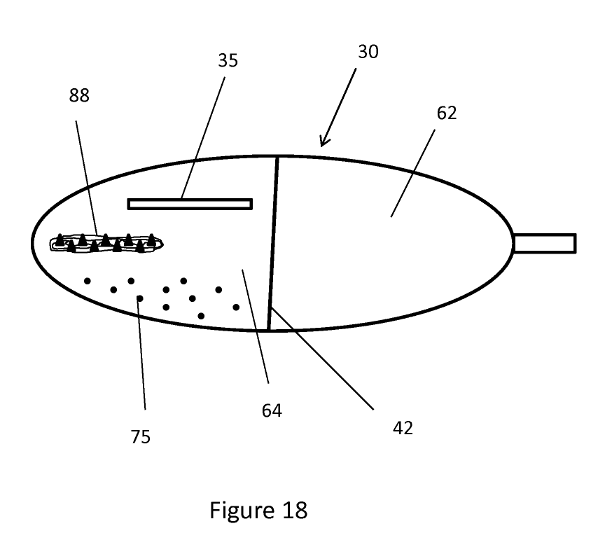

Another embodiment of the current invention of the blood products storage container is illustrated in FIG. 18. The container is divided into at least two sections with a porous membrane barrier between the sections. As shown in FIG. 18, the container 30 is divided in two zones 62 and 64. A porous membrane barrier 42 separates the two zones. One zone 64 contains adsorbent while the blood products are in zone 62. If concentrated platelets are put in zone 62, the plasma can penetrate through the membrane barrier 42 to be in direct contact with the adsorbents in zone 64. Platelets (or other blood cells) are confined in zone 62 because they could not pass through the membrane. As the container is placed on an oscillating platform, the plasma flushes through the membrane between the two zones. The pathogens with a size of about 1 to 2 micron (μm) are flushed through the membrane with the plasma. As the pathogens come in direct contact with the adsorbent media, it adheres to its surface and remains captured until it dies. The pathogens from zone 62 pass through the barrier to zone 64 to be captured by the adsorbents and die. After a short period of shaking the container on a cycling platform and allowing the plasma to flush in and out the two zones; most pathogens are moved in zone 64 to be captured and die. Therefore, the high majority of the pathogens inside the container are eliminated by the adsorbents. For illustration purposes the adsorbents in zone 64 are loose adsorbent particulates that could be activated carbon or porous resins 75, activated carbon cloth 35, or activated carbon matrix 88. These adsorbents can be used individually or in combination. These adsorbents can be coated by a biocompatible polymer such as hydrogel. The activated carbon 35 can be cloth, yarn, felt, paper, veil mat, felt rigid, or chop that could be supplied by “KRECA” Kureha America LLC. The activated carbon 35 can be woven or knitted cloth made of activated carbon fiber, micro fiber, nano fiber, or nano tube. The activated carbon 35 can be or compressed into a felt pad made of activated carbon fiber, micro fiber, nano fiber, or nano tube.

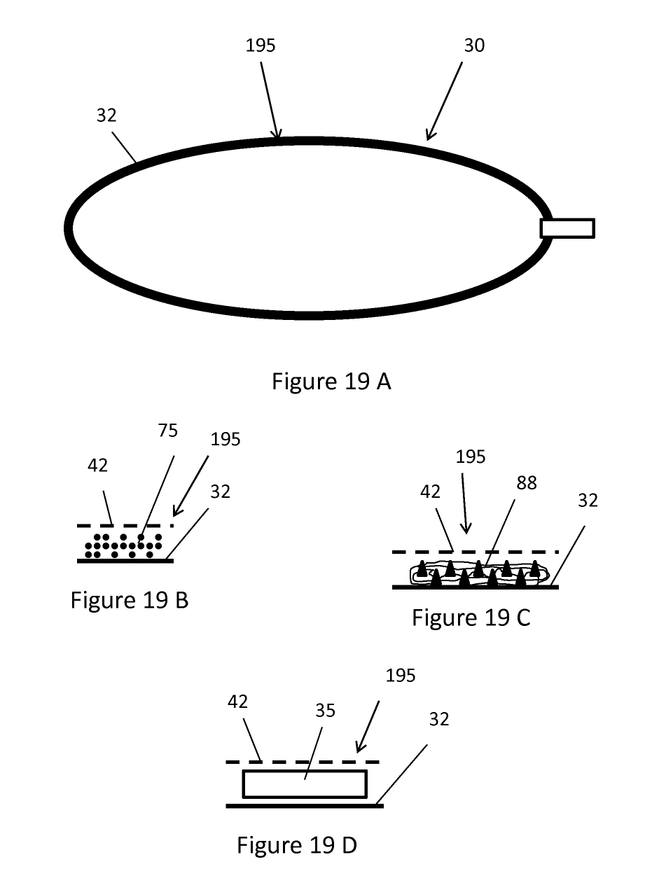

Another embodiment of the current invention of the blood products storage container is illustrated in FIGS. 19A-D. The container wall 195 is made of multiple layer encompassing outer layer 32, adsorbent layer, and porous membrane layer 42. As the container 30 is shaking on the cycling platform, the pathogens pass through the membrane layer with the plasma to contact the adsorbent layer. The pathogens are caught and eliminated by the adsorbent layer. Blood cells can't contact the adsorbent layer. Different laminated layers 195 of the container wall are illustrated in FIGS. 19B, 19C, and 19D. FIG. 19B illustrates container wall formed of outer layer 32 (outer-most layer), loose container layer 75 (adsorbent media layer), and porous membrane layer 42 (inner-most layer). FIG. 19C illustrates container wall formed of outer layer 32, adsorbent matrix layer 88, and porous membrane layer 42. FIG. 19D illustrates container wall formed of outer layer 32 (outer-most layer), activated carbon cloth 35 (adsorbent media layer), and porous membrane layer 42 (inner-most layer). The container wall can be built with multiple combinations of these layers. The porous membrane layer is not needed when the adsorbents are coated by a biocompatible polymer film such as hydrogel. The outer layer 32 is permeable to Oxygen and Carbon dioxide gases. The adsorbent layers of any style (loose adsorbent, matrix adsorbent, or carbon cloth coated or not) are permeable to Oxygen and Carbon dioxide gases. The porous membrane layer is permeable to Oxygen and Carbon dioxide gases. Therefore, the laminated wall is permeable to Oxygen and Carbon dioxide gases. It should be noted that the storage container can be built of laminated layers 195 sections and single layer wall sections (outer wall) 32. The laminated layer 195 does not need to cover the entire container outer wall. The activated carbon 35 can be woven or knitted cloth made of activated carbon fiber, micro fiber, nano fiber, or nano tube. The activated carbon 35 can be or compressed into a felt pad made of activated carbon fiber, micro fiber, nano fiber, or nano tube. The adsorbent media 75 could be made of activated carbon micro-fibers, nano fibaers, or nano tubes with high porosity and large surface area. It should be noted that the blood cell (platelet) storage bag can be placed during storage on any type of shaker, agitator, oscillator, and rotator. It can be placed on an oscillator platform or rotating drum. The platform can cycle in a linear reciprocating movement, cyclic movement, shaking movement, or orbital cycling movement. The frequency of the cycling movement can range from 1 cycle per minute to 600 cycles per minute. The frequency of the cycling movement can be of any value that would not damage or activate the stored blood cells including platelets during storage. The blood cell (including platelet) storage bag can be placed during storage on a stationary platform.

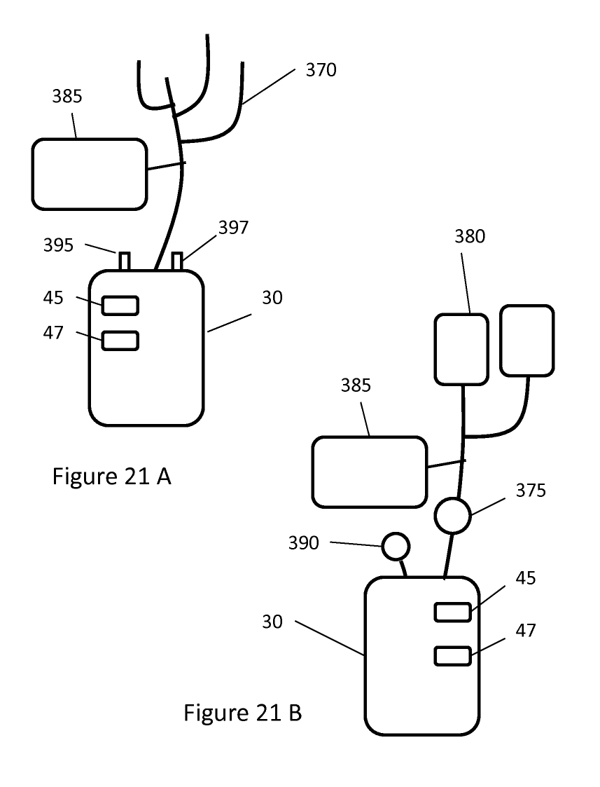

The storage container 30 can be integrated with any apheresis plastic disposable extracorporeal circuit. The container 30 can be also integrated with any dialysis plastic disposable extracorporeal circuit. Referring to FIG. 21A, the storage container 30 can be equipped with a set of tubing 370. These sterilized tubing are fluidly connected to the container. Platelet concentrate bags derived from whole blood unit 380 are connected to the tubing set 370 as shown in FIG. 21B. This tubing set permits a sterile fluid channel for the platelets to flow from bags 380 to container 30. Platelets from all the connected bags 380 can be driven to the container 30 where they are pooled. These bags 380 can be sterile connected to the tubing set 370 using (Terumo TCD) sterile weld device. In some cases, bags 380 could contain buffy coat fluid derived from whole blood unit. The storage container 30 can be integrated with a transfer bag 385 as shown in FIGS. 21A and 21B. This bag can be used to pool the platelets from bags 380 temporarily. The pooled platelet unit is then directed to the storage bag 30 and passing through a leukoreduction filter 375 that traps the white blood cells. Leukoreduced platelets are stored in bag 30 ready for storage or infusion. As shown in FIG. 21A, the storage container 30 can be integrated with an air vent 395 that is used to add sterile air to the platelet units and transfer bag to maximize platelet recovery. Also the storage container can be integrated with pH Safe Sensor 397 that is used for a sterile and noninvasive optical measurement of the stored fluid pH. The pH is determined by optically interpreting the biochemical changes occurring in the platelet solution as conducted by Blood Cell Storage, Inc. (454 North 34th St., Lower Level, Seattle, Wash. 98103, USA). As shown in FIG. 21B, the storage container 30 can be integrated with a sample pouch 390 to be used for bacteria detection for either pre-pool or final pool. The storage bag and the integrated tubing can be equipped with a number of clamps (not shown) and check valves (not shown). Bar codes and other machine readable techniques are used on the storage container and the integrated tubing harness for identification and documentation purposes.

FIGS. 21A and 21B demonstrate radio frequency (RF) labels 45 such as Texas Instrument Radio Frequency Identification labels (TIRFID) are used on the storage containers. These RF labels 45 can be used alone or in addition to a standard bar code labels. Other label type is magnetic strip labels 47. The RF labels are passive and emit radio frequency RF to identify the storage containers. Both RF and magnetic strip labels make it easy for blood banks to track all the platelets containers and correlate them to the original donor. This can be done for single donor platelet or for the pooled platelets. These labels are used to record information about the container and the stored platelets. Also information about the donor, the center, platelet preparation method, the start of the storage time, the elapsed storage time, and the original platelet count; all can be documented by these labels. This information can be retrieved instantly by RF reader antenna or magnetic strip reader.

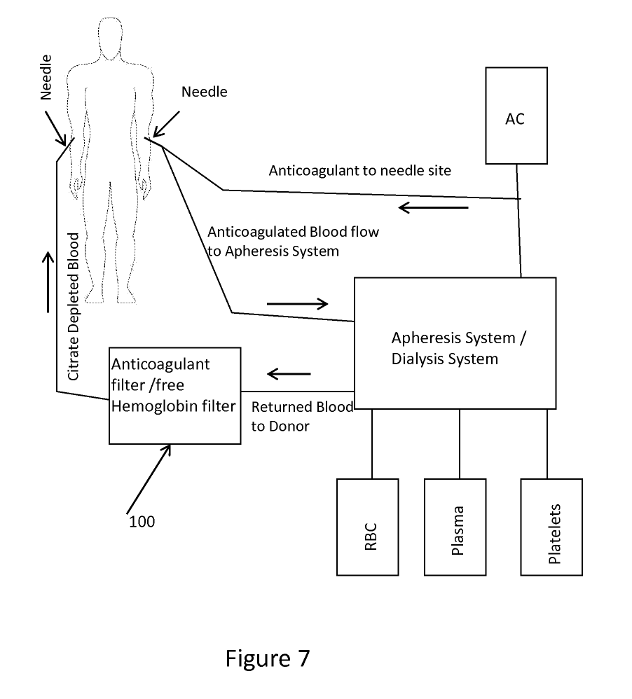

FIG. 7 depicting a schematic view of the connections between a donor and an apheresis system. The anticoagulant (AC) fluid is pumped to the vein puncture site to be mixed with the drawn blood at the needle. Typically, the AC flow line is hooked to a pump on the apheresis system in order to meter the exact ratio of AC to the drawn blood. This ratio for the apheresis system is 1:16 (by volume AC to blood) for most commonly used AC. As the drawn blood is mixed with AC it becomes resistant to clotting and it is called anticoagulated blood. The blood is processed by the apheresis system depending on the type of the system and the procedure. For example TerumoBCT Trima system has a protocol to remove platelets from the blood and store them in designated bags and return the rest of the blood components back to the donor. Fenwal Inc. Amicus system does the same. Haemonetics MCS8150 system and Fenwal Alyx system process the anticoagulated blood to remove the RBC's and store them in special bags while returning the rest of the blood components back to the donor. Another apheresis system is Haemonetics PCS system that processes the blood by removing the plasma and then returns the rest of the components back to the donor. In all these systems most of the AC that was mixed with the blood is infused into the donor with the returned blood components. FIG. 7 demonstrates a chamber containing ion exchange resins that is placed on the path of the returned blood components. As the apheresis system pumps the returned components back to the donor, the flow passes through a bed of ionic resins. These polymeric resins are weak basic, anion exchange resins that are specialized to react with weak acid solutions that have a pH level of 4.5 or greater to form a safe buffer. Therefore the effect of the citrate in the returned blood components solution is neutralized.

Referring to AABB (American Association of Blood Banks) Technical Manual 17th edition, the approved types of anticoagulants, their ratio to collected blood, and composition are listed in Table 2.

TABLE 2

| | CPD | CP2D | CPDA-1 | ACD-A | ACD-B | 4% Citrate |
|---|---|---|---|---|---|---|
| Variable | | | | | | |
| pH | 5.3-5.9 | 5.3-5.9 | 5.3-5.9 | 4.5-5.5 | 4.5-5.5 | 6.4-7.5 |
| Ratio | 1.4:10 | 1.4:10 | 1.4:10 | 1.5:10 | 2.5:10 | 0.625:10 |
| (ml solution/Blood) | | | | | | |
| Content | | | | | | |
| (mg in 63 ml solution) | | | | | | |
| Sodium Citrate | 1660 | 1660 | 1660 | 1386 | 832 | 2520 |
| Citric acid | 206 | 206 | 206 | 504 | 504 | As needed for |
| | | | | | | pH Adjustment |
| Dextrose | 1610 | 3220 | 2010 | 1599 | 956 | |
| Monobasic sodium | 140 | 140 | 140 | | | |
| phosphate | | | | | | |
| Adenine | 0 | 0 | 17.3 | | | |

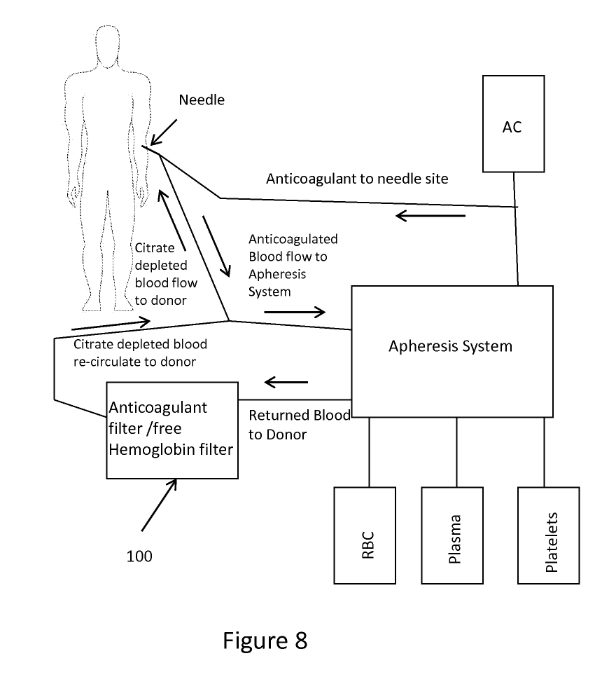

FIG. 8 depicts the configurations of the blood and blood components flow path between the donor and the apheresis system that utilizes one needle access for blood draw and return. The system ensures the looping of the return blood path to pass through the ionic resin exchange chamber before it is pumped to the donor.

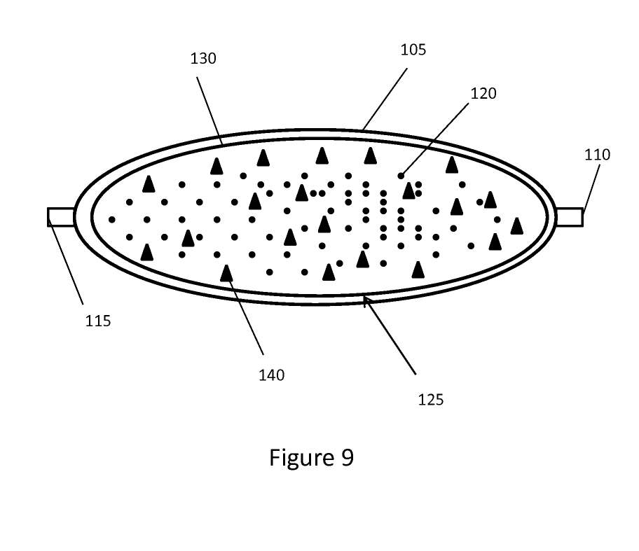

FIG. 9 shows a schematic configuration of a resin chamber (AC filter) 100. This chamber is designed to allow for the returned blood to flow through and thoroughly mix with the resin contained inside. The housing 105 of the chamber is made of material that can be sterilized. It could have a rigid, semi-rigid, stretchable, or flexible structure. The blood enters the chamber through the inlet port 110, and exits out through the outlet port 115. Resin particulates 120 are amassed inside a pouch 125 that is made of a screened mesh 130. Monofilament synthetic fibers can be woven very precisely to create textiles with narrow pore distribution. This precision weaving process creates fine mesh woven fabrics with apertures (hole sizes) as small as 1 micron. For the present invention, the screen mesh will have porosity of 100 μm to 200 μm enough to let the blood to pass freely through while trapping the resin inside. A 150μ mesh size is used as the size of the resin particulates range between (300 μm and 1,200 μm). These resins could have spherical shape. Activated carbon media 140 (particulates, spheres, or cloth) can be used with or without the resins 120 to adsorb free hemoglobin from the returned blood product. The activated carbon media can adsorb citrate from the returned blood products. The synthetic mesh could be made of polyester, polypropylene, or nylon materials and can be purchased from (Industrial Netting, 7681 Setzler Pkwy N., Minneapolis, Minn.) or from (SEFAR, 111 Calumet Street, Depew, N.Y.). The pouch 125 could be stretchable and can take the shape of the inner cavity of the chamber. The pouch is filled with resin (or activated carbon) that is selected for appropriate buffering of the acid solution. The volume of the resin is enough to handle all the solution that passes through the chamber. Returned blood components with citrate enter the chamber through inlet port 110. It flows through the resin mass or activated carbon mass inside the chamber 100. Ion exchange take place between the resin and the solution and some ions are absorbed by the resin or activated carbon media. The blood components solution is neutralized and becomes citrate free as it exits the chamber through port 115 and continues to be infused back into the donor.

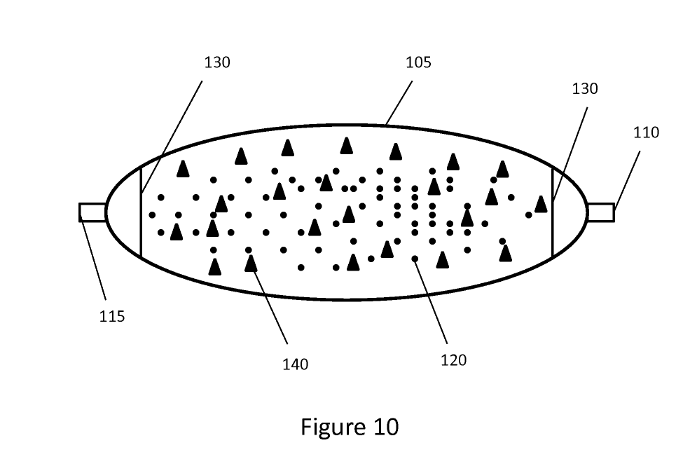

FIG. 10 depicts another configuration of the resin chamber 100. The ion exchange resin particulates (or activated carbon media 140) are stored inside the housing 105. Special screen mesh are placed between the inlet port 110 and the particulates 120 in order to confine the particulates inside the housing and prevent them from escaping through the inlet or outlet port.

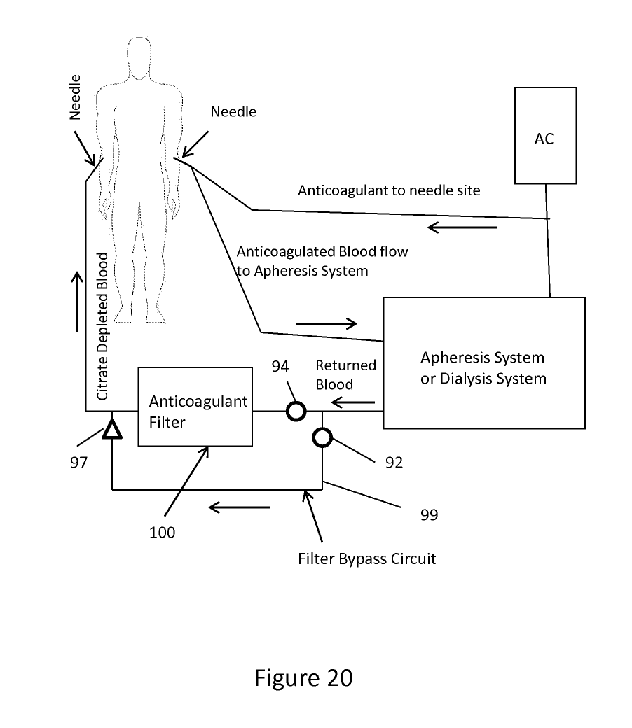

Referring to FIG. 7 the anticoagulant filter 100 is attached to the extracorporeal circuit in the section that extends from the apheresis system (or dialysis system) to the venipuncture site. Most specifically the anticoagulant filter (citrate filter) 100 is positioned as close as possible to the venipuncture (reinfusion) site. Although the system in FIG. 7 is labeled “Apheresis system” but it could be dialysis system also. FIG. 20 depicts an extracorporeal circuit for apheresis system or for a dialysis system with an anticoagulant filter bypass circuit 99. The returned blood flow from the apheresis (dialysis) system is first directed to pass through the anticoagulant filter 100. This is done by having valve 94 open and valve 92 closed. The anticoagulated blood follows its regular pass through valve 94 and filter 100 where it becomes citrate free (anticoagulant free), then it continues to the reinfusin needle site. The check valve 97 is added to the circuit to prevent the citrate depleted blood from returning back to the system through the bypass circuit. In this case the citrate is depleted from the blood before it is infused back in to the subject. Hence, the subject is saved from citrate toxicity. Toward the end of the batch procedure, valve 94 is closed and valve 92 is opened. Small amount of the anticoagulated blood is directed to the filter bypass circuit 99 and it continues to the reinfusin needle site. When the batch procedure is terminated and the blood flow is stopped, the portion of the extracorporeal circuit connecting the anticoagulant filter to the infusion needle site would be filled with anticoagulated blood. This will prevent the clotting of the stagnant blood and ensures its safety, as this portion of the blood will be infused in to the subject at the beginning of the consecutive batch.

Whole blood or concentrated red blood cell (RBC) are stored at (4° C.) for up to 42 days. Some of the red blood cells are hemolized or destroyed during storage causing the release of the intracellular hemoglobin to be mixed with the medium solution that is mostly plasma and some additive solution (glucose). When the blood is transfused to the patient, the free hemoglobin that was mixed with the plasma is infused into the patient body causing kidney toxicity. This problem is intensified with massive transfusion of many blood units or transfusion of aged blood. Therefore the removal of the free hemoglobin from the transfused blood to the patient would prevent serious complications that include multi-organ dysfunction, respiratory and renal insufficiency, and death.

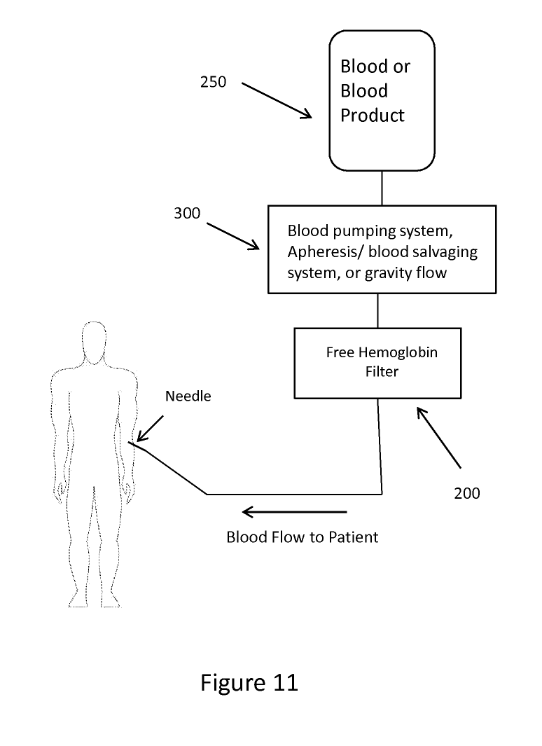

FIG. 11 depicting a schematic view of the operation of blood or blood component transfusion to a donor with a “Free Hemoglobin Filter” 200 attached to the transfusion set. The filter 200 is placed on the infused blood path to extract the free hemoglobin from the blood before it is infused in the patient's body. The blood or blood component is typically stored in a container or a reservoir 250 and it is directed to the patient by a transfusion set with a venipuncture needle. In some cases blood warming set is used in combination with the infusion set. The blood flow by gravity or it is pumped by special blood infusion systems 300 such as (Imed infusion pump—IMED Corp. 9775 Businesspark Avenue, San Diego, Calif., USA) or (Level 1—Smiths Medical 600 Cordwainer Drive, Norwell, Mass., USA).

It should be noted that filter 200 can also be used to extract prion from blood or blood product flow. Filter 200 also defined as “Prion Filter” removes Prion from blood. A prion is an infectious agent that is composed primarily of protein. Usually found in a mis-folded protein state. Prion has been implicated in a number of diseases in a variety of mammals. Human Prion Diseases are Creutzfeldt-Jakob Disease (CJD), Variant Creutzfeldt-Jakob Disease (vCJD), Gerstmann-Straussler-Scheinker Syndrome, Fatal Familial Insomnia, and Kuru. Animal Prion Diseases are Bovine Spongiform Encephalopathy (BSE), Chronic Wasting Disease (CWD), Scrapie, Transmissible mink encephalopathy, Feline spongiform encephalopathy, Ungulate spongiform encephalopathy. All known prion diseases affect the structure of the brain or other neural tissue, and all are currently untreatable and are always fatal.

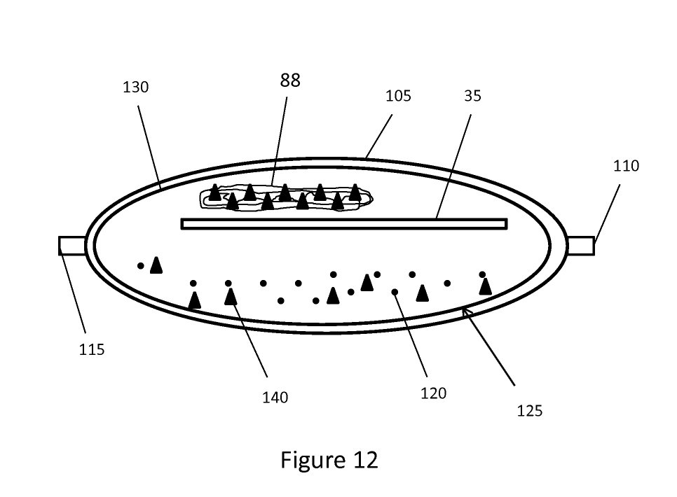

FIG. 12 depicts a schematic configuration of the free hemoglobin filter 200. This filter is designed to allow for the infused blood to flow through and thoroughly mix with the adsorbent media contained inside. The filter housing 105 is made of material that can be sterilized. It could have a rigid, semi-rigid, or flexible structure. The blood enters the filter housing through the inlet port 110, and exits out through the outlet port 115. Activated carbon (AC) media 140 are packed inside a pouch 125 that is made of a screened mesh 130. The screen mesh will have porosity of 100 μm to 300 μm enough to let the blood to pass freely through while trapping the AC media inside. The AC media 140 (particulates, spheres, or cloth) is used to adsorb free hemoglobin from the infused blood or blood product. The volume of the AC media inside the filter housing is enough to handle all the blood or RBC concentrate that passes through the filter. Infused blood with free hemoglobin (or prion) enters the filter housing through inlet port 110. It flows through the AC media that extract the free hemoglobin mixed with the blood plasma (or prion). The blood exiting the filter through the port 115 has no free hemoglobin (or prion) mixed with the plasma.

In another embodiment, porous resin particulates 120 can be used instead of the AC media inside the filter 200 to extract the free hemoglobin from the infused blood.

In another embodiment, porous resin particulates 120 can be used instead of the AC media inside the filter 200 to extract the free hemoglobin from the infused blood.

The adsorbent media (AC 140 or resin 120) could be coated with biocompatible layer such as hydrogel (pHEMA, PEG, or PVP), cellulose, silicone, or PMMA. The activated carbon 140 could be particulates, powder, pellets, spheres, rods, tubes, channels, chips, or cloth; coated or uncoated. The activated carbon could be impregnated with Silver, Zinc, or other materials to enhance its bactericide characteristics. The activated carbon could be coated with commonly used Silver Nitrate as antibacterial coating agent. The resin 120 could be styrene resin, Acrylic resins, ion exchange resins, or other porous resin.

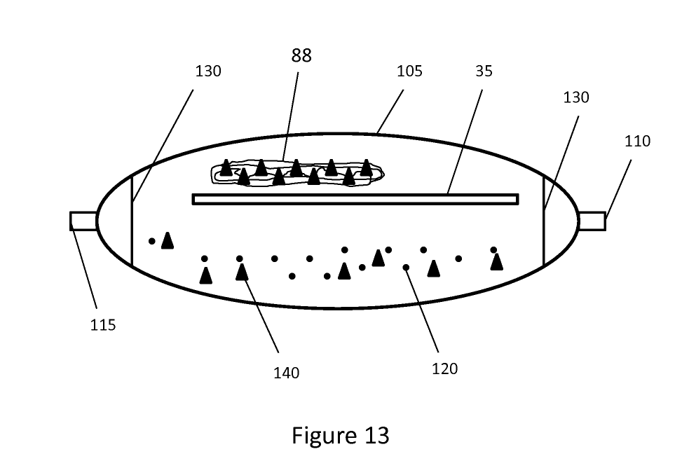

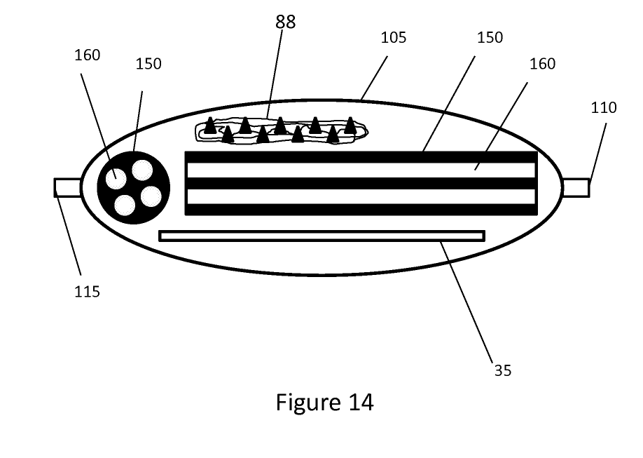

FIG. 13 demonstrates another embodiment of the current invention of the free hemoglobin filter. A screen 130 with a mesh size smaller than the adsorbent particulates used in the filter; is placed the filter housing 105 in the vicinity of the inlet port 110. A second screen 130 with a mesh size smaller than the adsorbent particulates used in the filter; is placed the filter housing 105 in the vicinity of the outlet port 115. The adsorbent media are placed inside the housing 105 and confined between these two screens in a way that no media particulate can pass through the screens or escape out of the filter housing through the outlet port or the inlet port. FIG. 14 demonstrates another embodiment of the current invention of the free hemoglobin filter. The adsorbent media in this style is configured in large volume structures that do not pass through the inlet or outlet ports; therefore there is no need for a screen to confine the media inside the housing. Examples of these adsorbent media structure are; activated carbon cloth 35, activated carbon or activated porous resin matrix 88, and activated carbon or porous resin structure 150 containing plurality of flow channels 160.

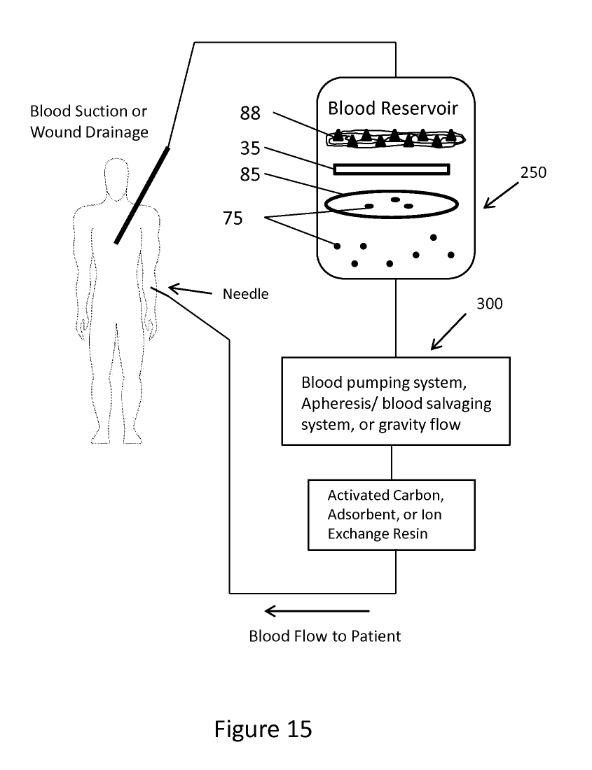

FIG. 15 demonstrates another concept for the extraction of the free hemoglobin from the infused blood. Adsorbent media is added to the blood reservoir or container 250 to extract the free hemoglobin before driving the blood to the infusion needle site. Different configurations of the adsorbent media inside the reservoir 250 are shown in FIG. 15. Loose adsorbent particulates 75 can be added to the container providing that a filter screen with appropriate mesh size (Not shown in the FIG. 15) is added at the exit port of the container to prevent any media particulate from exiting the container. The adsorbent media 75 could be confined inside a pouch made of a screened membrane material 85. The pouch prevents the media from exiting the container and flowing with the infused blood into the recipient body. Activated carbon cloth 32 can be added to the reservoir. This cloth can be covered by a biocompatible material membrane with porosity allows for plasma to pass through while preventing the blood cells from contacting the carbon surface. The cloth can be coated with biocompatible polymer such as hydrogel that allows the plasma to pass through while preventing the blood cells from contacting the carbon surface. Activated carbon matrix or porous resin matrix 88 can be added to the reservoir. All the adsorbent particulates are caught in the matrix that forms a large enough structure preventing it from exiting the reservoir. The matrix is made by having the adsorbent particulates 75 trapped in a netted polymeric string arrangement 77 that securely capture all the adsorbents in one structure 88 (as shown in FIG. 16). Or, it is made by generating a mesh of polymer stings formation with adhesive surface that securely bond to all adsorbents and holds them in one structure. The matrix can also be formed by capturing the adsorbent particulates 75 by a mesh of polymeric resin strings 77 covered with a lower melting temperature resin. The adsorbent particulates are mixed with these resin coated strings and heated to a temperature that melts the coating resin. The melted resin act as a welding agent that holds the adsorbents to the meshed strings therefore, forming a matrix structure. Activated carbon or porous resin structure 150 containing plurality of flow channels 160 as shown in FIG. 14 can be added to the blood reservoir shown in FIG. 15.

When stored blood units are added to the reservoir, the adsorbent media inside the reservoir extract the free hemoglobin from the blood. It is very common to have free hemoglobin mixed with the plasma of the stored red blood cell (RBC) concentrate units or whole blood units. As the whole blood or RBC concentrate unit is stored on shelves inside refrigerators at about 4° C., few red blood cells start to hemolize (damage to the cell membrane). The longer the blood is stored the more the possibility for the RBC to be hemolized. A 42 days aged blood unit could have considerable free hemoglobin (hemolysis) in it. Blood is salvaged by suction means during surgery (intra-operative) or by wound drainage means during recovery (post operative). The salvage blood is collected in reservoir to be re-infused back into the autologous patient. In some cases, salvaged blood is washed prior infusion to get rid of clots, lipid, and debris. Blood washing also helps in getting rid of the free hemoglobin. In other cases, salvaged blood is not washed. It is only filtered prior infusion by a screen mesh membrane to get rid of clots, lipid, and debris. When blood is salvaged by suction, the negative pressure gradient causes the blood cell to be hemolized. The suction pressure destroys the RBC and hence it generates high free hemoglobin levels. The same can be said about wound drainage where RBC cells have to flow in narrow perforated paths causing the cells to be destroyed.

The free hemoglobin filter of the current invention aims to reduce the risks associated with the transfusion of aged blood units. The hemoglobin adsorbent chamber of the current invention aims to reduce the risks associated with salvaged blood transfusion. The salvaged blood is the autologous blood that is collected from the bleeding patients during surgery (intra-operative) or during the recovery of surgery (post-operative). The blood is salvaged by wound suction or drainage and storing the blood in a temporary reservoir to be washed by special systems (such as Haemonetics, Braintree, Mass., USA; Cell Saver System) or to be filtered (not washed) before returning the blood to the patient. “Sangvia system” for intra-operative collection and reinfusion of autologous whole blood without washing is marketed by Wellspect HealthCare Company (P.O. Box 14, SE-431 21 Mölndal, Sweden). Another example of autologous blood salvage system that does not have the capability to wash the free hemoglobin from the salvaged blood is the “CBCII Blood Conservation System—ConstaVac” from Stryker (400 East Milham Avenue, Kalamazoo, Mich. 49001, USA).

The hemoglobin adsorbent chamber of the current invention aims to reduce the risks associated with massive blood transfusion of about 10 units or more of concentrated red blood cell units in 24 hours period. Massive blood transfusion is defined as the replacement of 50% of patient's blood volume in 12 to 24 hours. With the transfusion of such a large volume of blood in a short time leads to the infusion of a large volume of free hemoglobin that intoxicates the kidneys that causes death to the patient.

Having now described a few embodiments of the invention, it should be apparent to those skilled in the art that the foregoing is merely illustrative and not limiting, having been presented by way of example only. Numerous modifications and other embodiments are within the scope of ordinary skill in the art and are contemplated as falling within the scope of the invention as defined by the appended claims and equivalents thereto. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1) A container used for platelets storage having outer walls for fluid containment whereas said outer walls are permeable to gases such as O2 and CO2,
said container including one or more compartment extending from the inner surface of the outer wall and encompassing adsorbent media having a minimum surface area of 400 m²/g,
said compartment sharing one or more wall segments with the container outer wall and having other wall segments that are in direct contact with the stored fluid, said other wall segments are made of porous membranes that allow for fluid and pathogens to pass through while preventing blood cells including platelets from entering the compartment,
said container is agitated in a movement suitable for platelet storage, fluids stored inside the container pass through the porous membrane wall segments in and out of said compartments, pathogens suspended in the stored fluid pass through the pores inside the compartments and contact the adsorbent media, whereas said pathogens are captured by the adsorbent media and isolated inside the compartments, therefore reducing the pathogen concentration in the stored fluid that is kept outside the compartments, the pathogen depleted platelet concentrate solution that is kept outside the compartments is used for transfusion.

2) The platelet storage container defined in claim 1 wherein said adsorbent media is activated carbon particulates.

3) The platelet storage container defined in claim 1 wherein said adsorbent media is activated carbon fibers in woven cloth, knitted cloth, or felt cloth structure.

4) The platelet storage container defined in claim 1 wherein said adsorbent media is activated carbon nano-tubes.

5) The platelet storage container defined in claim 1 wherein said adsorbent media is porous polymeric resin beads of styrene or acrylic matrix.

6) The platelet storage container defined in claim 1 wherein said pathogens are killed by the adsorbent media on contact.

7) The platelet storage container defined in claim 1 wherein said adsorbent media is impregnated with Silver, Zinc, or other metal.

8) A container used for platelets storage having outer walls for fluid containment whereas said outer walls are permeable to gases such as O2 and CO2,
said container including one or more compartment extending from the inner surface of the outer wall and encompassing activated carbon fibers or nano tubes in woven cloth, knitted cloth, or felt cloth structure having minimum surface area of 400 m²/g,
said compartment sharing one or more wall segments with the container outer wall and having other wall segments that are in direct contact with the stored fluid, said other wall segments are made of porous membranes that allow for fluid and pathogens to pass through while preventing blood cells including platelets from entering the compartment,
said container is agitated in a movement suitable for platelet storage, fluids stored inside the container pass through the porous membrane wall segments in and out of said compartments, pathogens suspended in the stored fluid pass through the pores inside the compartments and contact activated carbon cloth, whereas said pathogens are captured by the activated carbon and isolated inside the compartments, therefore reducing the pathogen concentration in the stored fluid that is kept outside the compartments, the pathogen depleted platelet concentrate solution that is kept outside the compartments is used for transfusion.

9) The platelet storage container defined in claim 8 wherein said pathogens are killed by the activated carbon cloth on contact.

10) The platelet storage container defined in claim 8 wherein said compartments include porous polymeric resin beads of styrene or acrylic matrix in addition to the activated carbon fibers cloth.

11) The platelet storage container defined in claim 8 wherein said compartments include activated carbon particulates, nano tubes, or beads in addition to the activated carbon fibers cloth.

12) The platelet storage container defined in claim 8 wherein the cutting edges of the carbon cloth are sealed to prevent any disintegration of carbon fiber from the cloth structure.

13) The platelet storage container defined in claim 8 wherein the barrier membranes used in the compartments are made of polyester.

14) The platelet storage container defined in claim 8 wherein the activated carbon cloth is impregnated with Silver or other metals such as Zinc.

15) A container used for platelets storage having laminated outer walls for fluid containment whereas said laminated walls are permeable to gases such as O2 and CO2, Said laminated wall comprising an outermost layer impermeable to fluid and innermost layer having pores that allow for fluid and pathogens to pass through while acting as a barrier to blood cells, wherein at least one layer of adsorbent media having a minimum surface area of 400 m²/g is sandwiched between the innermost layer and the outermost layer,
fluids stored inside the container are in direct contact with the innermost layer of the laminated wall,
said container is agitated in a movement suitable for platelet storage, pathogens suspended in the stored fluid pass through the pores of the innermost layer and contact the adsorbent media layer, whereas said pathogens are captured by the adsorbent media layer, therefore reducing the pathogen concentration in the stored fluid, the pathogen depleted platelets that are kept away from the adsorbent media are used for human reinfusion.

16) The platelet storage container defined in claim 15 wherein said adsorbent media is activated carbon particulates or porous resin of styrene or acrylic matrix.

17) The platelet storage container defined in claim 15 wherein said adsorbent media is activated carbon fibers or nano tubes in woven cloth, knitted cloth, or felt cloth structure.

18) The platelet storage container defined in claim 15 wherein the innermost layer of the laminated wall is replaced by a biocompatible resin coating of the adsorbent media layer.

19) The platelet storage container defined in claim 15 wherein the innermost layer of the laminated wall is made of biocompatible polyester material.

20) The platelet storage container defined in claim 15 wherein said adsorbent media is activated carbon fibers or nano-tubes.
